(19) United States
(12) Patent Application Publication
(10) Pub. No.: US 2026/0226380 A1
(43) Pub. Date: Aug. 6, 2026

(54) KILLER YEASTS TO PREVENT SPOILAGE OF BEER

(71) Applicant: University of Idaho, Moscow, ID (US)

(72) Inventor: Paul A. Rowley, Moscow, ID (US)

(73) Assignee: University of Idaho, Moscow, ID (US)

(21) Appl. No.: 19/468,758

(22) Filed: Feb. 3, 2026

Related U.S. Application Data
(60) Provisional application No. 63/753,269, filed on Feb. 3, 2025.

(51) Int. Cl. C12C 11/00; C07K 14/395; C12N 1/18; C12N 7/00; C12N 15/81; C12R 1/865

(52) U.S. Cl. CPC C12C 11/003; C07K 14/395; C12N 1/18; C12N 7/00; C12N 15/81; C12C 2200/05; C12N 2710/00043; C12R 2001/865

(57) ABSTRACT

Variant K2 (K2v) killer toxins that inhibit the growth of diastatic yeast are provided. Also provided are engineered yeasts including K2v killer toxins, as well as methods of inhibiting diastatic yeast growth in fermented beverage products.

### CROSS REFERENCE TO RELATED APPLICATION

This claims the benefit of U.S. Provisional Application No. 63/753,269, filed Feb. 3, 2025, which is incorporated herein by reference in its entirety.

### ACKNOWLEDGMENT OF GOVERNMENT SUPPORT

This invention was made with government support under grant 2143405 awarded by U.S. National Science Foundation (NSF). The government has certain rights in the invention.

### FIELD

This application relates to yeast strains that inhibit diastatic yeast growth.

### INCORPORATION OF ELECTRONIC SEQUENCE LISTING

The Sequence Listing is submitted as an XML file named “Sequence.xml,” created on Jan. 30, 2026, 33,363 bytes, which is incorporated by reference herein.

### BACKGROUND

Spoilage of beer due to contamination by spoilage yeasts is a major concern for the brewing industry, but is not widely reported due to the cost of recalls and potential damage to brand image. The most damaging spoilage yeasts are strains of S. cerevisiae that express the STA1/2/3 family of glucanases. These yeasts are referred to as “diastatic yeasts.” Contamination by diastatic yeasts causes continued fermentation (over-attenuation) that can result in off flavors, gushing, and exploding packaging.

While the antimicrobial properties of hops can protect beers from bacterial spoilage, yeasts are more resistant. Pasteurization has long been the standard operating procedure for preventing spoilage, however, this technique is often not employed by craft breweries due to high capital costs. In addition, the high temperature zones of a pasteurizer contribute to premature beer aging and are perceived by breweries to negatively impact their beer sales. Thus, early diagnosis of diastatic yeast during the brewing process and destruction of contaminated products are the only current methods of control. New approaches, particularly those aimed to remediate or prevent contamination of diastatic yeast, are needed.

### SUMMARY

It is shown herein that growth of diastatic yeast can be inhibited by other yeast strains that produce antifungal proteins termed “killer toxins.” However, several diastatic yeast strains are resistant to canonical killer toxins, such as K1 and K2. After extensive screening, a K2 toxin variant (K2-variant; K2v) was found to be effective against diastatic yeast strains that are resistant to canonical killer toxins (e.g., K1 and/or K2).

Provided herein are engineered yeasts carrying a K2-variant (K2v) killer toxin. The K2v toxin includes one or more mutations relative to canonical K2 toxin, for example, a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259. In some aspects, the K2v toxin includes an amino acid sequence having at least 90% (e.g., 95%, 98%, 99%, etc.) sequence identity to SEQ ID NO: 1, or the K2v toxin includes or consists of SEQ ID NO: 1. The K2v toxin can be encoded on a vector, for example, a plasmid or viral vector. In some aspects, the engineered yeasts include a vector having at least 90% (e.g., 95%, 98%, 99%, etc.) sequence identity to SEQ ID NO: 3. In some aspects, the engineered yeasts include a recombinant vector comprising or consisting of SEQ ID NO: 3. In some aspects, the engineered yeasts are S. cerevisiae. In some aspects, the engineered yeasts are a brewer yeast (e.g., OYL090 (Omega Yeast San Diego Super), WLP001 (White Laps California Ale Yeast), CBC1 (Lallemand conditioning yeast), WLP028 (White Labs Edinburgh Scottish Ale), WLP940 (White Labs Mexican Lager Yeast), L28 (Imperial Yeast Urkel Czech lager yeast), OYL061 (Omega Yeast Voss Kveik), or OYL011).

Further provided are methods of inhibiting diastatic yeast growth in a fermented beverage product, including inoculating the fermented beverage product, or a pre-fermented form thereof (e.g., a wort), with an engineered yeast comprising an exogenous or heterologous nucleic acid molecule encoding a K2v toxin (e.g., an engineered yeast disclosed herein). In some aspects, the fermented beverage product is wine, cider, kombucha, or beer. In some aspects, the fermented beverage is beer. Inoculation can occur before or after fermentation. When inoculation occurs before fermentation, a pre-fermented form of the fermented beverage product is inoculated, for example, a mash or a wort. A fermented beverage product that is, or is suspected of being, contaminated with a diastatic yeast can be selected for use in a method disclosed herein. In some aspects, the contaminating diastatic yeast is resistant to canonical K2 toxin. In several aspects, the methods disclosed herein do not perceivably or negatively impact product flavor, stability, or final gravity of the fermented beverage product.

The foregoing and other features of this disclosure will become more apparent from the following detailed description of several aspects which proceeds with reference to the accompanying figures.

### BRIEF DESCRIPTION OF THE DRAWINGS

FIGS. 1A-1C. Diastatic yeasts are susceptible to canonical killer toxins produced by Saccharomyces yeasts. (FIGS. 1A-1B) Killer toxin activity against diastatic yeasts was qualitatively assessed based on the presence and size of growth inhibition zones and methylene blue staining around killer yeasts. Darker boxes on the cluster diagram represent a more prominent killer phenotype, whereas lighter boxes indicate little or no detectable killer phenotype. The non-killer yeast strains S. cerevisiae BY4741 (a laboratory strain) and WLP001 (a brewing yeast) were used as negative controls. (FIG. 1C) The antifungal activity of K1 and K2 against diastatic yeasts was quantified by measuring the total zone of growth inhibition (n=3). Error bars are standard deviation. Data from panel A is represented across each x-axis for comparison.

FIGS. 2A-2D. K2 killer toxin production by diastatic yeasts causes K2 resistance. (FIG. 2A) Killer toxin production by diastatic yeasts resistant to K1 and K2 killer toxins. (+) indicates a diastatic killer yeast capable of inhibiting the growth of S. cerevisiae BY4741. (−) indicates a non-killer yeast. (FIG. 2B) The extraction and analysis of dsRNAs from the diastatic yeasts by cellulose chromatography and gel electrophoresis. The detection of K1 or K2 genes by RT-PCR or PCR and using dsRNA or genomic DNA as templates. Genomic DNA extracted from S. cerevisiae with K1 or K2 genes integrated into the genome was used as a positive controls. (FIG. 2C) Exposure to cycloheximide (CHX) was used to cure diastatic strains of the killer phenotype due to the loss of dsRNA satellites as assayed by cellulose chromatography and RT-PCR. (FIG. 2D) Curing of dsRNAs resulted in increased susceptibility to K2 but not K1 as assayed on agar. Killer toxin activity against cured (CHX+) and wild-type (CHX−) diastatic yeasts was qualitatively assessed based on the presence and size of growth inhibition zones and methylene blue staining around either K1 or K2 killer yeasts (as described in FIGS. 1A-1C).

FIGS. 3A-3D. A novel killer toxin named K2v can inhibit the growth of diastatic yeasts resistant to K1 and K2 killer toxins. (FIG. 3A) Killer toxin activity against diastatic yeasts was qualitatively assessed based on the presence and size of growth inhibition zones and methylene blue staining around killer yeasts (as described in FIGS. 1A-1C). Killer toxins were expressed by wild-type killer yeasts or from killer toxin genes maintained on plasmids and expressed in the non-killer yeast strain BY4741. K2v was cloned from strain ACP. The non-killer yeast strain S. cerevisiae BY4741 was used as a negative control. (FIG. 3B) Representative agar plate killer assays of the sensitivity of K1- and K2-resistant diastatic yeasts to three novel killer yeasts. (FIG. 3C) Novel killer yeasts harbor totiviruses and M2 satellite dsRNAs, as confirmed by RT-PCR. (FIG. 3D) A linear representation of the secondary structure of the K2v killer toxin from strain ACP with mutations that alter the spectrum of antifungal activity when compared to K2. Cylinders and rectangles represent α-helices and β-sheets, respectively.

FIGS. 4A-4F. Killer yeasts can prevent hyperattenuation in fermentation trials. (FIG. 4A) Two ten-hectoliter (1,000 L) pioneer fermentation vessels were used for the fermentation trials (4A, left panel) with the brewing strain WLP-001, diastatic strain Belle Saison, and the K2 killer yeast strain Viva. A 100 L Esau Huber microprop yeast propagation plant was used to grow brewing and diastatic yeast strains (4A, right panel). The specific gravity (FIG. 4B), pH (FIG. 4C), and temperature (FIG. 4D) of the brewing trials were monitored for ~14 days with or without the addition of a K2 killer yeast. The dashed line in (FIG. 4D) represents the set point of 21° C. for cooling. Arrows indicate the addition of diastatic yeast after 7-8 days of fermentation with or without the addition of the K2 killer yeast strain. A spider diagram depicting tasting notes from the fermentation of (FIG. 4E) WLP-001 and (FIG. 4F) WLP-001 and diastatic yeast with or without the addition of a K2 killer yeast strain. Sensory characteristics are judged on a 10-point scale from absent (0) to high (10). Flavor notes are abbreviated as follows; Met (Metallic) EtOH (Alcohol), Iso (Iso-amyl acetate) Swe (Sweetness), HS (Hydrogen sulfide), Oxi (Oxidation/Papery), Malt (Malt character), Auto (Autolysis/Meaty) 4-Vg (4-vinyl guaiacol), Bit (Bitterness), Di (Diacetyl), DMS (Di-methyl sulfide), Fruit (Fruity/Esters).

FIGS. 5A-5B. K2 killer toxin production by diastatic yeasts causes K2 resistance. The killer phenotype of 14 diastatic yeasts on agar. (+) indicates a killer yeast capable of inhibiting the growth of S. cerevisiae BY4741. (−) indicates a non-killer yeast. The extraction and analysis of dsRNAs from the diastatic yeasts resistant to K1 and K2 killer toxins by cellulose chromatography and gel electrophoresis. The presence of K1 or K2 genes in purified dsRNAs was probed by RT-PCR or PCR and compared to genomic DNA extracted from strains with integrated K1 or K2 genes as positive controls.

FIG. 6. Diastatic killer yeasts cannot inhibit the growth of a K2 killer yeast. An agar plate killer assay with two diastatic killer yeast strains plated at high density atop a K2-producing killer yeast strain (S. cerevisiae CYC1172).

FIG. 7. Rare mating to create killer brewing strains of yeast. (left) Cell fusion of a prototrophic respiratory-deficient (p−) brewing strain with a kar1 auxotrophic (his3) K1 killer donor yeast strain with an M1 satellite creates a heterokaryon (the L-A totivirus that is essential for the maintenance of M1 is omitted for clarity). Selection for the brewing strain genotype with respiratory proficiency enables non-Mendelian inheritance of the K1 killer phenotype. (right) Representative image of K1 production K1 by the killer brewing strain and the strains used in rare mating.

FIG. 8. K1 killer toxin inhibits diastaticus in wort and has the same growth rate as the parent brewing strain. (left) Wort was used as a growth medium for a brewing yeast or its killer brewing yeast derivative. Spent growth media was filter-sterilized and used for growing diastaticus for three days (n=1). Cell counts were used to monitor the growth of yeast. (right) Doubling times of brewing and killer brewing strains of yeast. n.s.—not significant (n=3, T-test, p>0.05).

FIG. 9. The creation of a donor strain capable of receiving dsRNAs from killer yeasts by rare mating and cytoduction. Strains RD1 and RDT2 have the genotype MATα his4 kar1 ura3 ρ− and show an inability to grow on complete media lacking uracil with glycerol as a sole carbon source. BY4741 is a negative control for growth on complete media lacking uracil. The killer yeast CHD (labelled as K2v) is a K2v expressing strain that is a uracil prototroph and is respiratory proficient and is therefore capable of growth on complete media lacking uracil with glycerol as the sole carbon source.

FIG. 10. Killer toxin production by a new class of donor yeast strain. Strains RD1+K2v and RD1 T2+K2v have the genotype MATα his4 kar1 ura3 [L-A, M2v] and are capable of producing the K2v killer toxin the same as the parental strain CHD (labelled as K2v). Killer toxin production was assayed on YPD with pH 4.6 with methylene blue. As negative controls, the parental strains RD1 and RD1 T2 do not exhibit the killer phenotype.

### SEQUENCES

The nucleic and amino acid sequences listed herein are shown using standard letter abbreviations for nucleotide bases and amino acids. Only one strand of each nucleic acid sequence is shown, but the complementary strand is understood as included by any reference to the displayed strand.

SEQ ID NO: 1 is an exemplary amino acid sequence of K2v toxin.
MKETTTSLMQDELTLGEPATQARMCVRLLRFFIGLTITAFIIAACIIKSATGGSGYSKAVAVRG
EADTPSTIVGOLVERGGFQAWAVGAGIYLFAKIAYDTSKVTAAVCNPEALIAITSYVAYAPTLC
AGAYVIGAMSGAMSAGLALYAGYKGWOWGGPGGMAEREDVASFYSPLLNNTLYVGGDHTADYDS
ELATILGSVYNDVVHIGVYYDNSTGIVKRDSRPSMISWTVLHDNMMITSYHRPDOLGAAATAYK
AYTTNTTRVGKRQDGEWVSYSVYGENVDYERYPVAHLQEEADACYESLGNMITSQVQPCTQREC
YAMDQKVCAAVGFSSDAGVNSAMVGEAYFYAYGGVDGECDSG
SEQ ID NO: 2 is an exemplary nucleic acid sequence encoding
the K2v toxin of SEQ ID NO: 1.
ATGAAAGAGACTACCACCAGCCTGATGCAAGACGAGCTGACACTAGGTGAGCCGGCCACCCAAG
CGAGGATGTGCGTACGTCTATTACGTTTTTTCATAGGTCTGACTATAACCGCATTTATTATAGC
AGCCTGTATTATTAAAAGTGCGACAGGCGGTTCGGGATATTCTAAAGCAGTTGCTGTTCGGGGA
GAAGCGGACACCCCTTCCACAATTGTGGGCCAGCTCGTCGAGCGTGGCGGCTTCCAAGCTTGGG
CAGTGGGGGCTGGTATCTATTTGTTTGCCAAGATAGCATATGATACATCTAAGGTTACCGCAGC
TGTATGTAATCCGGAGGCGCTCATTGCTATCACATCGTATGTGGCATATGCCCCTACACTGTGT
GCTGGTGCATACGTTATTGGTGCCATGAGTGGGGCAATGTCGGCGGGCCTTGCTCTGTATGCCG
GTTACAAAGGATGGCAGTGGGGCGGCCCCGGGGGCATGGCAGAGAGAGAGGACGTGGCCTCTTT
TTATTCACCACTCCTGAACAACACTCTGTACGTGGGTGGGGACCACACTGCAGACTACGACAGT
GAATTGGCTACTATATTAGGTAGCGTATATAATGATGTGGTCCACCTGGGGGTGTATTACGATA
ACAGCACTGGAATTGTCAAGAGGGATTCGAGACCTAGCATGATCTCATGGACGGTGTTGCATGA
CAACATGATGATAACATCATACCATAGGCCAGACCAGCTGGGCGCAGCCGCGACAGCCTACAAA
GCTTATACCACAAACACAACACGGGTCGGTAAGAGGCAGGACGGTGAGTGGGTGTCATACTCGG
TCTACGGTGAGAATGTTGACTATGAAAGATACCCTGTAGCACATCTGCAAGAGGAGGCCGACGC
GTGTTACGAGAGTTTAGGTAATATGATTACGAGCCAGGTACAGCCCTGTACTCAGAGAGAATGT
TATGCTATGGATCAGAAAGTATGCGCAGCTGTCGGCTTCTCATCAGATGCGGGTGTTAACTCCG
CAATG
SEQ ID NO: 3 is an exemplary plasmid vector sequence
(pAG426-GPD-ccdB) encoding K2v.
GACGAAAGGGCCTCGTGATACGCCTATTTTTATAGGTTAATGTCATGATAATAATGGTTTCTTA
GTATGATCCAATATCAAAGGAAATGATAGCATTGAAGGATGAGACTAATCCAATTGAGGAGTGG
CAGCATATAGAACAGCTAAAGGGTAGTGCTGAAGGAAGCATACGATACCCCGCATGGAATGGGA
TAATATCACAGGAGGTACTAGACTACCTTTCATCCTACATAAATAGACGCATATAAGTACGCAT
TTAAGCATAAACACGCACTATGCCGTTCTTCTCATGTATATATATATACAGGCAACACGCAGAT
ATAGGTGCGACGTGAACAGTGAGCTGTATGTGCGCAGCTCGCGTTGCATTTTCGGAAGCGCTCG
TTTTCGGAAACGCTTTGAAGTTCCTATTCCGAAGTTCCTATTCTCTAGAAAGTATAGGAACTTC
AGAGCGCTTTTGAAAACCAAAAGCGCTCTGAAGACGCACTTTCAAAAAACCAAAAACGCACCGG
ACTGTAACGAGCTACTAAAATATTGCGAATACCGCTTCCACAAACATTGCTCAAAAGTATCTCT
TTGCTATATATCTCTGTGCTATATCCCTATATAACCTACCCATCCACCTTTCGCTCCTTGAACT
TGCATCTAAACTCGACCTCTACATTTTTTATGTTTATCTCTAGTATTACTCTTTAGACAAAAAA
ATTGTAGTAAGAACTATTCATAGAGTGAATCGAAAACAATACGAAAATGTAAACATTTCCTATA
CGTAGTATATAGAGACAAAATAGAAGAAACCGTTCATAATTTTCTGACCAATGAAGAATCATCA
ACGCTATCACTTTCTGTTCACAAAGTATGCGCAATCCACATCGGTATAGAATATAATCGGGGAT
GCCTTTATCTTGAAAAAATGCACCCGCAGCTTCGCTAGTAATCAGTAAACGCGGGAAGTGGAGT
CAGGCTTTTTTTATGGAAGAGAAAATAGACACCAAAGTAGCCTTCTTCTAACCTTAACGGACCT
ACAGTGCAAAAAGTTATCAAGAGACTGCATTATAGAGCGCACAAAGGAGAAAAAAAGTAATCTA
AGATGCTTTGTTAGAAAAATAGCGCTCTCGGGATGCATTTTTGTAGAACAAAAAAGAAGTATAG
ATTCTTTGTTGGTAAAATAGCGCTCTCGCGTTGCATTTCTGTTCTGTAAAAATGCAGCTCAGAT
TCTTTGTTTGAAAAATTAGCGCTCTCGCGTTGCATTTTTGTTTTACAAAAATGAAGCACAGATT
CTTCGTTGGTAAAATAGCGCTTTCGCGTTGCATTTCTGTTCTGTAAAAATGCAGCTCAGATTCT
TTGTTTGAAAAATTAGCGCTCTCGCGTTGCATTTTTGTTCTACAAAATGAAGCACAGATGCTTC
GTTCAGGTGGCACTTTTCGGGGAAATGTGCGCGGAACCCCTATTTGTTTATTTTTCTAAATACA
TTCAAATATGTATCCGCTCATGAGACAATAACCCTGATAAATGCTTCAATAATATTGAAAAAGG
AAGAGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGTGCACG
AGTGGGTTACATCGAACTGGATCTCAACAGCGGTAAGATCCTTGAGAGTTTTCGCCCCGAAGAA
CGTTTTCCAATGATGAGCACTTTTAAAGTTCTGCTATGTGGCGCGGTATTATCCCGTATTGACG
CCGGGCAAGAGCAACTCGGTCGCCGCATACACTATTCTCAGAATGACTTGGTTGAGTACTCACC
AGTCACAGAAAAGCATCTTACGGATGGCATGACAGTAAGAGAATTATGCAGTGCTGCCATAACC
ATGAGTGATAACACTGCGGCCAACTTACTTCTGACAACGATCGGAGGACCGAAGGAGCTAACCG
CTTTTTTGCACAACATGGGGGATCATGTAACTCGCCTTGATCGTTGGGAACCGGAGCTGAATGA
AGCCATACCAAACGACGAGCGTGACACCACGATGCCTGTAGCAATGGCAACAACGTTGCGCAAA
CTATTAACTGGCGAACTACTTACTCTAGCTTCCCGGCAACAATTAATAGACTGGATGGAGGCGG
ATAAAGTTGCAGGACCACTTCTGCGCTCGGCCCTTCCGGCTGGCTGGTTTATTGCTGATAAATC
TGGAGCCGGTGAGCGTGGGTCTCGCGGTATCATTGCAGCACTGGGGCCAGATGGTAAGCCCTCC
CGTATCGTAGTTATCTACACGACGGGGAGTCAGGCAACTATGGATGAACGAAATAGACAGATCG
CTGAGATAGGTGCCTCACTGATTAAGCATTGGTAACTGTCAGACCAAGTTTACTCATATATACT
TTAGATTGATTTAAAACTTCATTTTTAATTTAAAAGGATCTAGGTGAAGATCCTTTTTGATAAT
CTCATGACCAAAATCCCTTAACGTGAGTTTTCGTTCCACTGAGCGTCAGACCCCGTAGAAAAGA
TCAAAGGATCTTCTTGAGATCCTTTTTTTCTGCGCGTAATCTGCTGCTTGCAAACAAAAAAACC
ACCGCTACCAGCGGTGGTTTGTTTGCCGGATCAAGAGCTACCAACTCTTTTTCCGAAGGTAACT
GGCTTCAGCAGAGCGCAGATACCAAATACTGTTCTTCTAGTGTAGCCGTAGTTAGGCCACCACT
TCAAGAACTCTGTAGCACCGCCTACATACCTCGCTCTGCTAATCCTGTTACCAGTGGCTGCTGC
CAGTGGCGATAAGTCGTGTCTTACCGGGTTGGACTCAAGACGATAGTTACCGGATAAGGCGCAG
CGGTCGGGCTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCGTCGATTTTTGTGATGCT
CGTCAGGGGGGCGGAGCCTATGGAAAAACGCCAGCAACGCGGCCTTTTTACGGTTCCTGGCCTT
TTGCTGGCCTTTTGCTCACATGTTCTTTCCTGCGTTATCCCCTGATTCTGTGGATAACCGTATT
ACCGCCTTTGAGTGAGCTGATACCGCTCGCCGCAGCCGAACGACCGAGCGCAGCGAGTCAGTGA
GCGAGGAAGCGGAAGAGCGCCCAATACGCAAACCGCCTCTCCCCGCGCGTTGGCCGATTCATTA
ATGCAGCTGGCACGACAGGTTTCCCGACTGGAAAGCGGGCAGTGAGCGCAACGCAATTAATGTG
AGTTAGCTCACTCATTAGGCACCCCAGGCTTTACACTTTATGCTTCCGGCTCGTATGTTGTGTG
GAATTGTGAGCGGATAACAATTTCACACAGGAAACAGCTATGACCATGATTACGCCAAGCGCGC
AATTAACCCTCACTAAAGGGAACAAAAGCTGGAGCTCAGTTTATCATTATCAATACTCGCCATT
TCAAAGAATACGTAAATAATTAATAGTAGTGATTTTCCTAACTTTATTTAGTCAAAAAATTAGC
CTTTTAATTCTGCTGTAACCCGTACATGCCCAAAATAGGGGGGGGGTTACACAGAATATATAAC
ATCGTAGGTGTCTGGGTGAACAGTTTATTCCTGGCATCCACTAAATATAATGGAGCCCGCTTTT
TAAGCTGGCATCCAGAAAAAAAAAGAATCCCAGCACCAAAATATTGTTTTCTTCACCAACCATC
AGTTCATAGGTCCATTCTCTTAGCGCAACTACAGAGAACAGGGGCACAAACAGGCAAAAAACGG
GCACAACCTCAATGGAGTGATGCAACCTGCCTGGAGTAAATGATGACACAAGGCAATTGACCCA
CGCATGTATCTATCTCATTTTCTTACACCTTCTATTACCTTCTGCTCTCTCTGATTTGGAAAAA
GCTGAAAAAAAAGGTTGAAACCAGTTCCCTGAAATTATTCCCCTACTTGACTAATAAGTATATA
AAGACGGTAGGTATTGATTGTAATTCTGTAAATCTATTTCTTAAACTTCTTAAATTCTACTTTT
ATAGTTAGTCTTTTTTTTAGITTTAAAACACCAGAACTTAGTTTCGACGGATTCTAGAACTAGT
GGATCCCCCATCACAAGTTTGTACAAAAAAGCAGGCTCCGAATTCGCCCTTATGAAAGAGACTA
CCACCAGCCTGATGCAAGACGAGCTGACACTAGGTGAGCCGGCCACCCAAGCGAGGATGTGCGT
ACGTCTATTACGTTTTTTCATAGGTCTGACTATAACCGCATTTATTATAGCAGCCTGTATTATT
AAAAGTGCGACAGGCGGTTCGGGATATTCTAAAGCAGTTGCTGTTCGGGGAGAAGCGGACACCC
CTTCCACAATTGTGGGCCAGCTCGTCGAGCGTGGCGGCTTCCAAGCTTGGGCAGTGGGGGCTGG
TATCTATTTGTTTGCCAAGATAGCATATGATACATCTAAGGTTACCGCAGCTGTATGTAATCCG
GAGGCGCTCATTGCTATCACATCGTATGTGGCATATGCCCCTACACTGTGTGCTGGTGCATACG
TTATTGGTGCCATGAGTGGGGCAATGTCGGCGGGCCTTGCTCTGTATGCCGGTTACAAAGGATG
GCAGTGGGGCGGCCCCGGGGGCATGGCAGAGAGAGAGGACGTGGCCTCTTTTTATTCACCACTC
CTGAACAACACTCTGTACGTGGGTGGGGACCACACTGCAGACTACGACAGTGAATTGGCTACTA
TATTAGGTAGCGTATATAATGATGTGGTCCACCTGGGGGTGTATTACGATAACAGCACTGGAAT
TGTCAAGAGGGATTCGAGACCTAGCATGATCTCATGGACGGTGTTGCATGACAACATGATGATA
ACATCATACCATAGGCCAGACCAGCTGGGCGCAGCCGCGACAGCCTACAAAGCTTATACCACAA
ACACAACACGGGTCGGTAAGAGGCAGGACGGTGAGTGGGTGTCATACTCGGTCTACGGTGAGAA
TGTTGACTATGAAAGATACCCTGTAGCACATCTGCAAGAGGAGGCCGACGCGTGTTACGAGAGT
TTAGGTAATATGATTACGAGCCAGGTACAGCCCTGTACTCAGAGAGAATGTTATGCTATGGATC
AGAAAGTATGCGCAGCTGTCGGCTTCTCATCAGATGCGGGTGTTAACTCCGCAATGGTCGGTGA
GGCCTACTTCTATGCCTATGGTGGGGTTGATGGTGAATGTGACAGCGGCTAGAAGGGCGAATTC
GACCCAGCTTTTTTCTTGTACAAAGTGGTGATGGGCTGCAGGAATTCGATATCAAGCTTATCGA
TACCGTCGACCTCGAGTCATGTAATTAGTTATGTCACGCTTACATTCACGCCCTCCCCCCACAT
CCGCTCTAACCGAAAAGGAAGGAGTTAGACAACCTGAAGTCTAGGTCCCTATTTATTTTTTTAT
AGTTATGTTAGTATTAAGAACGTTATTTATATTTCAAATTTTTCTTTTTTTTCTGTACAGACGC
GTGTACGCATGTAACATTATACTGAAAACCTTGCTTGAGAAGGTTTTGGGACGCTCGAAGGCTT
TAATTTGCGGCCGGTACCCAATTCGCCCTATAGTGAGTCGTATTACGCGCGCTCACTGGCCGTC
GTTTTACAACGTCGTGACTGGGAAAACCCTGGCGTTACCCAACTTAATCGCCTTGCAGCACATC
CCCCTTTCGCCAGCTGGCGTAATAGCGAAGAGGCCCGCACCGATCGCCCTTCCCAACAGTTGCG
CAGCCTGAATGGCGAATGGACGCGCCCTGTAGCGGCGCATTAAGCGCGGCGGGTGTGGTGGTTA
CGCGCAGCGTGACCGCTACACTTGCCAGCGCCCTAGCGCCCGCTCCITTCGCTTTCTTCCCTTC
CTTTCTCGCCACGTTCGCCGGCTTTCCCCGTCAAGCTCTAAATCGGGGGCTCCCTTTAGGGTTC
CGATTTAGTGCTTTACGGCACCTCGACCCCAAAAAACTTGATTAGGGTGATGGTTCACGTAGTG
GGCCATCGCCCTGATAGACGGTTTTTCGCCCTTTGACGTTGGAGTCCACGTTCTTTAATAGTGG
ACTCTTGTTCCAAACTGGAACAACACTCAACCCTATCTCGGTCTATTCTTTTGATTTATAAGGG
ATTTTGCCGATTTCGGCCTATTGGTTAAAAAATGAGCTGATTTAACAAAAATTTAACGCGAATT
TTAACAAAATATTAACGCTTACAATTTCCTGATGCGGTATTTTCTCCTTACGCATCTGTGCGGT
ATTTCACACCGCATAGGGTAATAACTGATATAATTAAATTGAAGCTCTAATTTGTGAGTTTAGT
ATACATGCATTTACTTATAATACAGTTTTTTAGTTTTGCTGGCCGCATCTTCTCAAATATGCTT
CCCAGCCTGCTTTTCTGTAACGTTCACCCTCTACCTTAGCATCCCTTCCCTTTGCAAATAGTCC
TCTTCCAACAATAATAATGTCAGATCCTGTAGAGACCACATCATCCACGGTTCTATACTGTTGA
CCCAATGCGTCTCCCTTGTCATCTAAACCCACACCGGGTGTCATAATCAACCAATCGTAACCTT
CATCTCTTCCACCCATGTCTCTTTGAGCAATAAAGCCGATAACAAAATCTTTGTCGCTCTTCGC
AATGTCAACAGTACCCTTAGTATATTCTCCAGTAGATAGGGAGCCCTTGCATGACAATTCTGCT
AACATCAAAAGGCCTCTAGGTTCCTTTGTTACTTCTTCTGCCGCCTGCTTCAAACCGCTAACAA
TACCTGGGCCCACCACACCGTGTGCATTCGTAATGTCTGCCCATTCTGCTATTCTGTATACACC
CGCAGAGTACTGCAATTTGACTGTATTACCAATGTCAGCAAATTTTCTGTCTTCGAAGAGTAAA
AAATTGTACTTGGCGGATAATGCCTTTAGCGGCTTAACTGTGCCCTCCATGGAAAAATCAGTCA
AGATATCCACATGTGTTTTTAGTAAACAAATTTTGGGACCTAATGCTTCAACTAACTCCAGTAA
TTCCTTGGTGGTACGAACATCCAATGAAGCACACAAGTTTGTTTGCTTTTCGTGCATGATATTA
AATAGCTTGGCAGCAACAGGACTAGGATGAGTAGCAGCACGTTCCTTATATGTAGCTTTCGACA
TGATTTATCTTCGTTTCCTGCAGGTTTTTGTTCTGTGCAGTTGGGTTAAGAATACTGGGCAATT
TCATGTTTCTTCAACACTACATATGCGTATATATACCAATCTAAGTCTGTGCTCCTTCCTTCGT
TCTTCCTTCTGTTCGGAGATTACCGAATCAAAAAAATTICAAGGAAACCGAAATCAAAAAAAAG
AATAAAAAAAAAATGATGAATTGAAAAGGTGGTATGGTGCACTCTCAGTACAATCTGCTCTGAT
GCCGCATAGTTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTT
TTCACCGTCATCACCGAAACGCGCGA
SEQ ID NO: 4 is the canonical amino acid sequence of K2 toxin.
MKETTTSLVQDELTLGEPATRARMCVRLLRFFIGLTITAFIIAACIIKSATGGSGYSNAVAVRG
EADTPSTIVGOLVERGGFQAWAVGAGIYLFAKIAYDTSKVTAAVCNPEALIAITSYVAYAPTLC
AGAYVIGAMSGAMSAGLALYAGYKGWOWSGPGGMAEREDVASFYSPLLNNTLYVGGDHTADYDS
ELATILGSVYNDVVHLGVYYDNSTGIVKRDSRPSMTSWTVLHDNMMITSYHRPDOLGAAATAYK
AYATNTTRVGKRQDGEWVSYSVYGENVDYERYPVAHLQEEADACYESLGNMITSQVQPCTQREC
YAMDQKVCAAVGFSSDAGVNSAMVGEAYFYAYGGVDGECDSG
SEQ ID NO: 5 is an exemplary nucleic acid sequence encoding
the canonical K2 toxin of SEQ ID NO: 4.
ATGAAAGAGACTACCACCAGCCTGGTGCAAGACGAGCTGACACTAGGTGAGCCGGCCACCCGAG
CAAGGATGTGCGTACGTCTATTACGTTTTTTCATAGGTCTGACTATAACCGCATTTATTATAGC
AGCCTGTATTATTAAAAGTGCGACAGGCGGTTCGGGATATTCTAATGCAGTTGCTGTTCGGGGA
GAAGCGGACACCCCTTCCACAATTGTGGGCCAGCTCGTCGAGCGTGGCGGCTTCCAAGCTTGGG
CAGTGGGGGCTGGTATCTATTTGTTTGCCAAGATAGCATATGATACATCTAAGGTTACCGCAGC
TGTATGTAATCCGGAGGCGCTCATTGCTATCACATCGTATGTGGCATATGCCCCTACACTGTGT
GCTGGTGCATACGTTATTGGTGCCATGAGTGGGGCAATGTCGGCGGGCCTTGCTCTGTATGCCG
GTTACAAAGGATGGCAGTGGAGCGGCCCCGGGGGCATGGCAGAGAGAGAGGACGTGGCCTCTTT
TTATTCACCACTCCTGAACAACACTCTGTACGTGGGTGGGGACCACACTGCAGACTACGACAGT
GAATTGGCTACTATATTAGGTAGCGTATATAATGATGTGGTCCACCTGGGGGTGTATTACGATA
ACAGCACTGGAATTGTCAAGAGGGATTCGAGACCTAGCATGACCTCATGGACGGTGTTGCATGA
CAACATGATGATAACATCATACCATAGGCCAGACCAGCTGGGCGCAGCCGCGACAGCCTACAAA
GCTTATGCCACAAACACAACACGGGTCGGTAAGAGGCAGGACGGTGAGTGGGTGTCATACTCGG
TCTACGGTGAGAATGTTGACTATGAAAGATACCCTGTAGCACATCTGCAAGAGGAGGCCGACGC
GTGTTACGAGAGTTTAGGTAATATGATTACGAGCCAGGTACAGCCCTGTACTCAGAGAGAATGT
TATGCTATGGATCAGAAAGTATGCGCAGCTGTCGGCTTCTCATCAGATGCGGGTGTTAACTCCG
CAATGGTCGGTGAGGCCTACTTCTATGCCTATGGTGGGGTTGATGGTGAATGTGACAGCGGCTA
G
SEQ ID NOs: 6-17 are primers to amplify the full-length K1, K2
and K2v genes.
SEQ ID NO: 18 is an exemplary vector(pAG306-GPD-ccdB) including
K2v.
tcgcgcgtttcggtgatgacggtgaaaacctctgacacatgcagctcccggagacggtcacagc
ttgtctgtaagcggatgccgggagcagacaagcccgtcagggcgcgtcagcgggtgttggcggg
tgtcggggctggcttaactatgcggcatcagagcagattgtactgagagtgcaccacgcttttc
aattcaattcatcattttttttttattcttttttttgatttcggtttctttgaaatttttttga
ttcggtaatctccgaacagaaggaagaacgaaggaaggagcacagacttagattggtatatata
cgcatatgtagtgttgaagaaacatgaaattgcccagtattcttaacccaactgcacagaacaa
aaacctgcaggaaacgaagataaatcatgtcgaaagctacatataaggaacgtgctgctactca
tcctagtcctgttgctgccaagctatttaatatcatgcacgaaaagcaaacaaacttgtgtgct
tcattggatgttcgtaccaccaaggaattactggagttagttgaagcattaggtcccaaaattt
gtttactaaaaacacatgtggatatcttgactgatttttccatggagggcacagttaagccgct
aaaggcattatccgccaagtacaattttttactcttcgaagacagaaaatttgctgacattggt
aatacagtcaaattgcagtactctgcgggtgtatacagaatagcagaatgggcagacattacga
atgcacacggtgtggtgggcccaggtattgttagcggtttgaagcaggcggcagaagaagtaac
aaaggaacctagaggccttttgatgttagcagaattgtcatgcaagggctccctatctactgga
gaatatactaagggtactgttgacattgcgaagagcgacaaagattttgttatcggctttattg
ctcaaagagacatgggtggaagagatgaaggttacgattggttgattatgacacccggtgtggg
tttagatgacaagggagacgcattgggtcaacagtatagaaccgtggatgatgtggtctctaca
ggatctgacattattattgttggaagaggactatttgcaaagggaagggatgctaaggtagagg
gtgaacgttacagaaaagcaggctgggaagcatatttgagaagatgcggccagcaaaactaaaa
aactgtattataagtaaatgcatgtatactaaactcacaaattagagcttcaatttaattatat
cagttattaccctgcggtgtgaaataccgcacagatgcgtaaggagaaaataccgcatcaggaa
attgtaaacgttaatattttgttaaaattcgcgttaaatttttgttaaatcagctcatttttta
accaataggccgaaatcggcaaaatcccttataaatcaaaagaatagaccgagatagggttgag
tgttgttccagtttggaacaagagtccactattaaagaacgtggactccaacgtcaaagggcga
aaaaccgtctatcagggcgatggcccactacgtgaaccatcaccctaatcaagttttttggggt
cgaggtgccgtaaagcactaaatcggaaccctaaagggagcccccgatttagagcttgacgggg
aaagccggcgaacgtggcgagaaaggaagggaagaaagcgaaaggagcgggcgctagggcgctg
gcaagtgtagcggtcacgctgcgcgtaaccaccacacccgccgcgcttaatgcgccgctacagg
gcgcgtcgcgccattcgccattcaggctgcgcaactgttgggaagggcgatcggtgcgggcctc
ttcgctattacgccagctggcgaaggggggatgtgctgcaaggcgattaagttgggtaacgcca
gggttttcccagtcacgacgttgtaaaacgacggccagtgaattgtaatacgactcactatagg
gcgaattggagctctagtacggattagaagccgccgagcgggcgacagccctccgacggaagac
tctcctccgtgcgtcctcgtcttcaccggtcgcgttcctgaaacgcagatgtgcctcgcgccgc
actgctccgaacaataaagattctacaatactagcttttatggttatgaagaggaaaaattggc
agtaacctggccccacaaaccttcaaattaacgaatcaaattaacaaccataggatgataatgc
gattagttttttagccttatttctggggtaattaatcagcgaagcgatgatttttgatctatta
acagatatataaatggaaaagctgcataaccactttaactaatactttcaacattttcagtttg
tattacttcttattcaaatgtcataaaagtatcaacaaaaaattgttaatatacctctatactt
taacgtcaaggagaaaaaaccccggattctagaactagtggatcccccGTACAAAAAAGCAGGC
TCCGAATTCGCCCTTATGAAAGAGACTACCACCAGCCTGATGCAAGACGAGCTGACACTAGGTG
AGCCGGCCACCCAAGCGAGGATGTGCGTACGTCTATTACGTTTTTTCATAGGTCTGACTATAAC
CGCATTTATTATAGCAGCCTGTATTATTAAAAGTGCGACAGGCGGTTCGGGATATTCTAAAGCA
GTTGCTGTTCGGGGAGAAGCGGACACCCCTTCCACAATTGTGGGCCAGCTCGTCGAGCGTGGCG
GCTTCCAAGCTTGGGCAGTGGGGGCTGGTATCTATTTGTTTGCCAAGATAGCATATGATACATC
TAAGGTTACCGCAGCTGTATGTAATCCGGAGGCGCTCATTGCTATCACATCGTATGTGGCATAT
GCCCCTACACTGTGTGCTGGTGCATACGTTATTGGTGCCATGAGTGGGGCAATGTCGGCGGGCC
TTGCTCTGTATGCCGGTTACAAAGGATGGCAGTGGGGCGGCCCCGGGGGCATGGCAGAGAGAGA
GGACGTGGCCTCTTTTTATTCACCACTCCTGAACAACACTCTGTACGTGGGTGGGGACCACACT
GCAGACTACGACAGTGAATTGGCTACTATATTAGGTAGCGTATATAATGATGTGGTCCACCTGG
GGGTGTATTACGATAACAGCACTGGAATTGTCAAGAGGGATTCGAGACCTAGCATGATCTCATG
GACGGTGTTGCATGACAACATGATGATAACATCATACCATAGGCCAGACCAGCTGGGCGCAGCC
GCGACAGCCTACAAAGCTTATACCACAAACACAACACGGGTCGGTAAGAGGCAGGACGGTGAGT
GGGTGTCATACTCGGTCTACGGTGAGAATGTTGACTATGAAAGATACCCTGTAGCACATCTGCA
AGAGGAGGCCGACGCGTGTTACGAGAGTTTAGGTAATATGATTACGAGCCAGGTACAGCCCTGT
ACTCAGAGAGAATGTTATGCTATGGATCAGAAAGTATGCGCAGCTGTCGGCTTCTCATCAGATG
CGGGTGTTAACTCCGCAATGAAGGGCGAATTCGACCCAGCgatgggctgcaggaattcgatatc
aagcttatcgataccgtcgacctcgagtcatgtaattagttatgtcacgcttacattcacgccc
tccccccacatccgctctaaccgaaaaggaaggagttagacaacctgaagtctaggtccctatt
tatttttttatagttatgttagtattaagaacgttatttatatttcaaatttttcttttttttc
tgtacagacgcgtgtacgcatgtaacattatactgaaaaccttgcttgagaaggttttgggacg
ctcgaaggctttaatttgcggccggtacccagcttttgttccctttagtgagggttaattccga
gcttggcgtaatcatggtcatagctgtttcctgtgtgaaattgttatccgctcacaattccaca
caacataggagccggaagcataaagtgtaaagcctggggtgcctaatgagtgaggtaactcaca
ttaattgcgttgcgctcactgcccgctttccagtcgggaaacctgtcgtgccagctgcattaat
gaatcggccaacgcgcggggagaggcggtttgcgtattgggcgctcttccgcttcctcgctcac
tgactcgctgcgctcggtcgttcggctgcggcgagcggtatcagctcactcaaaggcggtaata
cggttatccacagaatcaggggataacgcaggaaagaacatgtgagcaaaaggccagcaaaagg
ccaggaaccgtaaaaaggccgcgttgctggcgtttttccataggctcggcccccctgacgagca
tcacaaaaatcgacgctcaagtcagaggtggcgaaacccgacaggactataaagataccaggcg
ttcccccctggaagctccctcgtgcgctctcctgttccgaccctgccgcttaccggatacctgt
ccgcctttctcccttcgggaagcgtggcgctttctcaatgctcacgctgtaggtatctcagttc
ggtgtaggtcgttcgctccaagctgggctgtgtgcacgaaccccccgttcagcccgaccgctgc
gccttatccggtaactatcgtcttgagtccaacccggtaagacacgacttatcgccactggcag
cagccactggtaacaggattagcagagcgaggtatgtaggcggtgctacagagttcttgaagtg
gtggcctaactacggctacactagaaggacagtatttggtatctgcgctctgctgaagccagtt
accttcggaaaaagagttggtagctcttgatccggcaaacaaaccaccgctggtagcggtggtt
tttttgtttgcaagcagcagattacgcgcagaaaaaaaggatctcaagaagatcctttgatctt
ttctacggggtctgacgctcagtggaacgaaaactcacgttaagggattttggtcatgagatta
tcaaaaaggatcttcacctagatccttttaaattaaaaatgaagttttaaatcaatctaaagta
tatatgagtaaacttggtctgacagttaccaatgcttaatcagtgaggcacctatctcagcgat
ctgtctatttcgttcatccatagttgcctgactgcccgtcgtgtagataactacgatacgggag
ggcttaccatctggccccagtgctgcaatgataccgcgagacccacgctcaccggctccagatt
tatcagcaataaaccagccagccggaagggccgagcgcagaagtggtcctgcaactttatccgc
ctccatccagtctattaattgttgccgggaagctagagtaagtagttcgccagttaatagtttg
cgcaacgttgttgccattgctacaggcatcgtggtgtcacgctcgtcgtttggtatggcttcat
tcagctccggttcccaacgatcaaggcgagttacatgatcccccatgttgtgaaaaaaagcggt
tagctccttcggtcctccgatcgttgtcagaagtaagttggccgcagtgttatcactcatggtt
atggcagcactgcataattctcttactgtcatgccatccgtaagatgcttttctgtgactggtg
agtactcaaccaagtcattctgagaatagtgtatgcggcgaccgagttgctcttgcccggcgtc
aatacgggataataccgcgccacatagcagaactttaaaagtgctcatcattggaaaacgttct
tcggggcgaaaactctcaaggatcttaccgctgttgagatccagttcgatgtaacccactcgtg
cacccaactgatcttcagcatcttttactttcaccagcgtttctgggtgagcaaaaacaggaag
gcaaaatgccgcaaaaaagggaataagggcgacacggaaatgttgaatactcatactcttcctt
tttcaatattattgaagcatttatcagggttattgtctcatgagcggatacatatttgaatgta
tttagaaaaataaacaaataggggttccgcgcacatttccccgaaaagtgccacctgacgtcta
agaaaccattattatcatgacattaacctataaaaataggcgtatcacgaggccctttcgtc

### DETAILED DESCRIPTION

### I. Introduction

The rising popularity of craft beers and the growth of the craft brewing industry means that more beer is being produced in facilities lacking pasteurization. Pasteurization stabilizes beer against contamination by spoilage organisms, including yeast and bacteria. The shift away from pasteurization could be due to the high capital costs of pasteurization equipment and increased energy and water usage. Additionally, the dominant beer styles produced by the craft brewing industry are negatively affected by the high temperature of the process. Beer styles that have been aggressively hopped post-boil, such as India Pale Ales (IPAs), will suffer from excessive exposure to oxygen when pasteurized (Kaneda et al., Int J Food Sci Technology 29:195-200). This treatment is perceived to increase the rate of staling and can lead to off-putting flavors described as papery, wet, cardboard-like, leathery, or even “catty.” This means that many breweries avoid pasteurizing as it causes the degradation of delicate hop aromas. However, aversion to pasteurization can increase the risk of spoilage. Although the antimicrobial properties of hops can protect beers from bacterial spoilage, yeasts are more resistant and represent a more problematic spoilage organism without pasteurization.

One group of yeasts that cause spoilage in craft breweries are Saccharomyces cerevisiae strains that express the STA1 gene to produce an extracellular glucoamylase enzyme. These strains are referred to as diastatic yeasts, a name that is derived from diastase, an alternative nomenclature for amylase. Diastatic yeasts are an evolutionarily related group of S. cerevisiae strains used commercially to produce high-gravity Belgian-style beers (Peter et al., Nature 556:339-344; Krogerus et al., Appl Microbiol Biot 104:3745-3756). Diastatic yeasts are unique because the STA1 gene allows the hydrolysis of long-chain polysaccharides such as dextrin and starch. In non-Belgian-style beer, these carbohydrates remain after the primary fermentation has consumed simple di- and monosaccharides created in the mashing process. Dextrins and starches are usually unavailable to most commercial brewing strains as they lack the appropriate hydrolytic enzymes to break the glycosidic linkages between carbohydrate monomers.

The STA1 gene evolved due to a fusion of FLO11 and SGA1 (Tamaki et al., Mol Gen Genetics Mgg 164:205-209; Yamashita et al., J Bacteriol 161:574-582). The gene fusion resulted in a chimeric protein with the N-terminus of the FLO11 gene joined to almost the complete open reading frame of the SGA1 glucoamylase. The 5′ end of FLO11 fused to SGA1 enabled the transport of Sta1 into the extracellular milieu, where it can hydrolyze residual dextrin and starch to glucose monomers. The resulting glucose is used to prolong fermentation (usually after packaging), referred to as over-, super-, or hyperattenuation.

Hyperattenuation results in the overproduction of CO₂ and alcohol, imparting off flavors and promoting “gushing” and the explosion of containers. The STA1 gene is also part of a family undergoing gene duplications and translocations, creating the paralogs STA2 and STA3 (Pretorius et al., Curr Genet 14:9-13). Significantly, the overall occurrence of spoilage by diastatic yeasts in Europe increased between 2008 and 2017 and is an important problem for the brewing industry worldwide (M-Dörnberg et al., Master Brewers Association of the Americas Technical Quarterly 54:140-148; Latorre et al., Brewing Science 73:51-57).

Good hygiene, strain husbandry, and monitoring practices can reduce the likelihood of contamination by diastatic yeasts. However, viable cell counts on agar-based media can be somewhat unreliable and take days after sampling to identify contaminants (Burns et al., J Am Soc Brew Chem 79:167-180). The gold-standard molecular methods for rapidly detecting the STA1 gene by PCR require specialized equipment, reagents, and personnel to perform and interpret these assays (Navarro et al., Foods 9:1373). Even if diastatic yeast contamination is detected, the primary course of action in a brewery without a pasteurizer is the destruction of the contaminated product. Therefore, there is an urgent need to develop cost-effective technologies that actively prevent or remediate contamination by diastatic yeasts.

Killer yeasts can produce extracellular proteinaceous killer toxins that inhibit the growth of competing species of fungi (Schmitt et al., Nat Rev Microbiol 4:212-221; Schaffrath et al., Physiology and Genetics. The Mycota (A Comprehensive Treatise on Fungi as Experimental Systems for Basic and Applied Research); Marquina et al., International Microbiology doi.org/10.1007/s10123-002-0066-z). Studies have shown effectiveness of killer yeasts in preventing spoilage of fruits, silage, and wine fermentation (see, e.g., Jijakli et al., JOBC wprs Bulletin 25:29-32; Kitamoto et al., Journal of Dairy Science 76:803-811; Liu et al., Int J Food Microbiol 131:280-282; Lowes et al., Applied and Environmental Microbiology 66:1066-1076; Plantania et al., Food Microbiology 30:219-225; Santos et al., Microbiology (Reading) 150:2527-2534; Perez et al., PloS one 11:e0165590-21; Diaz et al., Microorganisms 8:1477). These successes have led to the application of certain fungal species and genetically engineered crops as biological controls in agricultural processes (see, Quijano et al., Biotechnology Reports 11:90-98; Schlaich et al., Plant Biotechnology Journal 4:63-75; Haissam et al., Antonie van Leeuwenhoek 99:93-105; Schnurer et al., Antonie van Leeuwenhoek 99:5-12).

Saccharomyces yeasts were some of the first species identified as producing killer toxins (Bevan et al., p. 202-203. In. Proc. Int. Congr. Genet), and surveys have estimated that many strains of S. cerevisiae are killer yeasts (Vijayraghavan et al., G3: Genes, Genomes, Genet doi.org/10.1093/g3journal/jkad167; Crabtree et al., FEMS Yeast Res 23:foad046). Killer toxin expression often depends on cytoplasmic double-stranded RNAs (dsRNAs) replicated and encapsidated by viruses of the family Totiviridae (Wickner et al., Adv Virus Res 86:1-36). To date, nine dsRNA-encoded killer toxins are produced by different strains of S. cerevisiae (K1, K2, K28, and Klus) and S. paradoxus (K62, K1L, K21, K74, K21/K66) (Dignard et al., Mol Gen Genet 227:127-136; Schmitt et al., Virology 213:341-351; Rodriguez-Cousino et al., Appl Environ Microbiol 77:1822-1832; Bostian et al., Cell 36:741-751; Rodriguez-Cousino et al., Toxins 9:313-20; Rodriguez-Cousino et al., Appl Environ Microb e02030-21; Fredericks et al., Plos Genet 17: e1009341; Vepštaitė-Monstavičė et al., Viruses 10). At least two functional genome-encoded killer toxins exist in S. cerevisiae (KHR and KHS) (Goto et al., Agr Biol Chem Tokyo 55:1953-1958; Goto et al., Agr Biol Chem Tokyo 54:979-984). These killer toxins share little primary sequence homology and can target susceptible cells by different mechanisms, such as by disrupting cell membranes (K1, K1L, and K2) or by arresting the cell cycle (K28) (Martinac et al., Proc Natl Acad Sci 87:6228-6232; Schmitt et al., Microbiology 142:2655-2662). The antifungal activities of killer toxins are generally limited to closely related species, and there is evidence of widespread resistance across different yeast lineages, which has limited their potential as broad-spectrum antifungals (Buzzini et al., FEMS Yeast Res 7:749-760; Golubev et al., Mycocins (Killer Toxins), p. 1-8. In Kurtzman, C P, Fell, J W (eds.), 4th ed).

This study demonstrates that killer toxins from S. cerevisiae have potent antifungal activity against different strains of diastatic yeasts. After screening an extensive collection of Saccharomyces killer yeast, diastatic strains resistant to canonical killer toxins were found to be susceptible to a non-canonical variant of the K2 toxin referred to herein as K2v. As proof-of-principle for applying killer yeasts to control diastatic contamination, a K2 killer yeast strain was able to prevent hyperattenuation in an industrial-scale fermentation with no adverse effect on the final gravity. This work provides a framework for using killer yeasts in the craft brewing industry to prevent future losses and lawsuits.

The use of killer toxins to prevent diastatic contamination is a unique and innovative approach and is a radical departure from the status quo, which is focused on early detection of diastatic yeasts.

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a K2v toxin” includes singular or plural toxins and can be considered equivalent to the phrase “at least one K2v toxin.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated.

Although many methods and materials similar or equivalent to those described herein can be used, particular suitable methods and materials are disclosed herein. In case of conflict, the present specification, including explanations of terms, will control. In addition, the materials, methods, and examples are illustrative only and not intended to be limiting. To facilitate review of the various aspects of the disclosure, the following explanations of terms are provided:

About: Unless context indicates otherwise, “about” refers to plus or minus 5% of a reference value. For example, “about” 100 refers to 95 to 105.

Amino Acid Substitution: The replacement of an amino acid in a polypeptide with one or more different amino acids. In some aspects, amino acid substitution is 1:1 (each amino acid that is substituted in a polypeptide is replaced with a single different amino acid). In the context of a protein sequence, an amino acid substitution can also be referred to as a mutation.

Beer: A carbonated, alcoholic beverage made from fermenting cereal grain, particularly malted-barley. The grain is mashed to convert starch to sugars, which dissolve in water to form the wort. Fermentation of the wort by yeast, particularly Saccharomyces cerevisiae, produces ethanol and carbonation in the beer. Most modern beer is brewed with hops, which act as a flavoring agent as well as a natural preservative and stabilizer. Beer can also be brewed with other flavoring agents, including herbs and fruit.

Conservative Variant: A nucleic acid or amino acid sequence containing a conservative variation (e.g., a conservative amino acid substitution) relative to a reference sequence. Conservative variations do not significantly affect or decrease function of a corresponding protein. Significance can refer to a desired p-value from a statistical test, for example, a p-value of less than 0.05 or less than 0.01, when activity of a conservative variant is compared to the reference sequence. Individual variations, including substitutions, deletions or additions, that alter, add or delete a single amino acid or a small percentage of amino acids (for instance less than 5%, in some aspects less than 1%) in an encoded sequence are also conservative variations when the alterations do not significantly affect or decrease function of a protein.

A conservative amino acid substitution is a substitution of an amino acid with another that is chemically similar. The following six groups are examples of amino acids that are considered conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are substitutions with an amino acid that is not considered chemically similar. Non-conservative substitutions may alter or reduce activity or function of the protein.

Control: A reference standard. In some aspects, the control is a negative control (e.g., a sample known to lack diastatic yeast). In other aspects, the control is a positive control (e.g., a sample known to contain diastatic yeast). In still other aspects, the control is a standard reference value or range of values that represent a baseline or normal result.

A difference between a test sample and a control can be an increase or conversely a decrease. The difference can be a qualitative difference or a quantitative difference, for example a statistically significant difference (e.g., a p-value of less than 0.05 or less than 0.01). In some examples, a difference is an increase or decrease, relative to a control, of at least about 5%, for example, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

CRISPR/Cas System: CRISPR (Clustered Regularly Interspaced Short Palindromic Repeats)/Cas (CRISPR-associated) systems, or CRISPR systems, use RNA-guided nucleases termed CRISPR-associated or “Cas” endonucleases (e.g., Cas9) to cleave a target nucleotide sequence. In a typical CRISPR/Cas system, a Cas endonuclease is directed to a target nucleotide sequence (e.g., a site in the genome that is to be sequence-edited) by a sequence-specific, non-coding “guide RNA” (gRNA). A double stranded DNA break induced by a CRISPR system can be repaired through homology-directed repair (HDR) using an artificial donor nucleic acid (repair template). In molecular biology applications, HDR mechanisms can be used to facilitate site-specific gene/genome editing; for example, a double stranded break can be induced in a target sequence via a CRISPR/Cas, and an artificial repair template containing a desired nucleotide sequence can be integrated or “knocked-in” by HDR at the location of the double-stranded break.

Target sequences must generally be adjacent to a “protospacer adjacent motif” (“PAM”) that is specific for a given Cas endonuclease; however, PAM sequences are short and relatively non-specific, and thus frequently appear throughout a given genome.

Degenerate Variant: In the context of the present disclosure, a “degenerate variant” refers to a variant nucleic acid sequence due to redundancy of the genetic code. Despite having different nucleotide sequences, degenerate variants all encode the same amino acid sequence. This is possible because most amino acids are specified by more than one codon.

Diastatic Yeast: Strains of Saccharomyces cerevisiae that produce an extracellular glucoamylase encoded by a STA gene, particularly STA1, however the STA family also includes STA2 and STA3. Sequence information of exemplary STA genes are publicly available, for example, on GenBank® (see, e.g., Sta1 (CAA26487.1) and Sta2 (P29760.1)).

Exogenous: Originating from outside a referenced organism. For example, a nucleic acid molecule that is exogenous to Saccharomyces cerevisiae is a nucleic acid that originated from a source other than the organism itself (e.g., a synthetic nucleic acid molecule introduced into Saccharomyces cerevisiae). A cell including an exogenous nucleic acid is not, or otherwise excludes, any naturally occurring cell or organism.

Expression: Transcription or translation of a nucleic acid sequence. For example, a gene is expressed when its DNA is transcribed into RNA, which in some examples is processed to become mRNA. A gene may also be expressed when its mRNA is translated into an amino acid sequence, such as a protein. In a particular example, a heterologous gene is expressed when it is transcribed into an RNA. In another example, a heterologous gene is expressed when its RNA is translated into an amino acid sequence.

Expression Control Sequence: A nucleic acid sequence that regulates the expression of another nucleic acid sequence to which it is operatively linked. Expression control sequences can include, but are not limited to, promoters, enhancers, transcription terminators, internal ribosomal entry sites (IRES), start codons (e.g., ATG) in protein-encoding genes, splicing signals, spacers for maintaining correct reading frame for protein translation of mRNA, and stop codons. The term “control sequences” is intended to include, at a minimum, components whose presence can influence expression, and can also include additional components whose presence is advantageous, for example, leader sequences.

Expression control sequences can include a promoter. A promoter is a minimal sequence sufficient to direct transcription. Also included are promoter elements that are sufficient to render promoter-dependent gene expression controllable for cell-type specific, tissue-specific, or inducible by external signals or agents; such elements may be located in the 5′ or 3′ regions of a nucleic acid that is to be expressed.

Expression Vector: A vector including expression control sequences operatively linked to a nucleotide sequence to be expressed. An expression vector includes sufficient cis-acting elements for expression in a host; other elements for expression can be supplied by the host cell or in an in vitro expression system. Expression vectors include all useful expression vectors known in the art, including cosmids, plasmids (e.g., naked or contained in liposomes) and viruses (e.g., lentiviruses, retroviruses, adenoviruses, and adeno-associated viruses). Useful expression vectors for yeast have been described, for example, in Alberti et al. “A suite of gateway cloning vectors for high-throughput genetic analysis in Saccharomyces cerevisiae.” Yeast 24:913-919, 2007.

Fermented Beverage Product: A beverage product made by a process that includes fermentation with a brewer yeast, for example, Saccharomyces cerevisiae. Exemplary fermented beverage products include, but are not limited to, beer, wine, cider, and kombucha. In some aspects, a fermented beverage product is beer.

Fusion Protein: A protein containing amino acid sequences from at least two different proteins or peptides. To create a fusion protein, the nucleic acid sequences are typically fused in the same reading frame and do not contain any internal stop codons. Fusion proteins, particularly short fusion proteins, can also be generated by chemical synthesis.

Heterologous: Originating from a different genetic source.

Host Cell: A cell in which a vector can be propagated and its nucleic acid expressed. The cell may be prokaryotic or eukaryotic. In some aspects, the host is a yeast cell. The term also includes any progeny of the subject host cell. It is understood that all progeny may not be identical to the parental cell since there may be mutations that occur during replication. However, such progeny are included when the term “host cell” is used to the extent that the progeny are functionally equivalent to parental cells.

Increase or Decrease: A positive (increase) or negative (decrease) difference relative to a reference value, such as a control. The difference can be qualitative or quantitative. In some examples, the difference is statistically significant (e.g., P-Value less than 0.05 or 0.01). In some aspects, the difference is an increase relative to a control of at least 5%, such as at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 500%, or greater than 500%. In some aspects, the difference is a decrease relative to a control of at least 5%, such as at least 10%, at least 20%, at least 30%, at least 40%, at least 50%, at least 60%, at least 70%, at least 80%, at least 90%, at least 95%, at least 98%, or 100%.

Isolated or Purified: An “isolated” or “purified” biological component (for example, a nucleic acid, protein, or cell) is one that has been substantially separated from other biological components in the environment in which the component occurs, e.g., separated from other chromosomal and extra-chromosomal DNA and RNA, proteins and/or cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids. An “isolated” microorganism (e.g., a recombinant Saccharomyces cerevisiae) has been substantially separated or purified away from microorganisms of different types, strains, or species. Microorganisms can be isolated by a variety of techniques, such as serial culturing and/or using selection markers conferring resistance to certain chemicals, for example, antibiotics.

Absolute purity or isolation is not required, it is intended as a relative term. Thus, for example, a purified/isolated protein, nucleic acid, or cell preparation is one in which the protein, nucleic acid, or cell is more enriched than the protein, nucleic acid, or cell was in its initial environment. In one example, a preparation is purified/isolated such that the protein, nucleic acid, or cell represents at least 50% of the total content of the preparation, such as at least 60%, at least 70%, at least 80%, at least 90%, at least 95%, at least 98%, or at least 99% of the total content of the preparation.

K2v: A variant of the canonical K2 toxin (see, SEQ ID NO: 4). While several variant K2 sequences may exist, as used herein, K2v refers to a specific sequence that includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259, wherein the amino acid positions refer to the K2v sequence of SEQ ID NO: 1. In some aspects, K2v includes or consists of SEQ ID NO: 1, or a conservative variant thereof.

Killer Toxins: Extracellular proteinaceous toxins produced by fungi that inhibit competing fungi. Killer toxin expression often depends on cytoplasmic double-stranded RNAs (dsRNAs) replicated and encapsidated by viruses of the family Totiviridae. To date, nine dsRNA-encoded killer toxins have been described in strains of S. cerevisiae (K1, K2, K28, and Klus) and S. paradoxus (K62, K1L, K21, K74, and K21/K66). In addition, at least two functional genome-encoded killer toxins exist in S. cerevisiae (KHR and KHS). Killer toxins share little primary sequence homology and can target susceptible cells by different mechanisms, such as by disrupting cell membranes (K1, K1L, and K2) or by arresting the cell cycle (K28). However, there is evidence of widespread resistance across different yeast lineages, which has limited their potential use as broad-spectrum antifungals.

Linker: A molecule that links two separate molecules into one contiguous molecule. Non-limiting examples of peptide linkers include glycine-serine peptide linkers. Unless context indicates otherwise, reference to “linking” a first polypeptide and a second polypeptide, or to two polypeptides “linked” together, or to a first polypeptide having a “linkage” to a second polypeptide, refers to covalent linkage by peptide bond (for example via a peptide linker) such that the first and second polypeptides form a contiguous polypeptide chain. If a peptide linker is involved, the covalent linkage of the first and second polypeptides can be to the N- and C-termini of the peptide linker. Typically, such linkage is accomplished using molecular biology techniques to genetically manipulate DNA encoding the first polypeptide linked to the second polypeptide by the peptide linker.

Modified or Mutated: A nucleic acid or protein having one or more changes in sequence relative to a reference sequence (e.g., wild-type). Sequence modifications include, for example, substitutions, insertions, and deletions, or combinations thereof. For proteins, insertions include amino and/or carboxyl terminal fusions as well as intrasequence insertions of single or multiple amino acid residues. Insertions for nucleic acid sequence include 5′ or 3′ additions or intrasequence insertions of single or multiple nucleotides. Deletions are characterized by the removal of one or more amino acid residues from a reference protein sequence or one or more nucleotides from a reference nucleic acid sequence. Substitutions are those in which at least one amino acid residue or nucleotide has been removed and a different residue or nucleotide inserted in its place.

Substitutions, deletions, insertions or any combination thereof may be combined to arrive at a final modified sequence. Protein modifications can be prepared by modification of nucleotides in the DNA encoding the protein, thereby producing DNA encoding the modification. Techniques for making insertion, deletion, or substitution modifications at predetermined sites in DNA or RNA are known.

Original Gravity: In beer-making, gravity refers to the relative density (compared to water) of a liquid during the brewing process. The original gravity refers to a measure of the fermentable and unfermentable substances present in the wort before fermentation. Increasing values of original gravity generally indicate higher availability of fermentable substances that yeasts can convert to alcohol. The measurement of wort gravity is useful as an indicator of the potential alcoholic strength of the beer. Gravity is typically measured in Degrees Plato (° P), although some brewers use the Balling scale. The “final gravity” refers to °P measured after fermentation.

Operably Linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Promoter: A nucleic acid control sequence that directs transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. Constitutive promoters are often used to overexpress a nucleic acid in a host. In contrast, the activity of an “inducible promoter” is regulated by an external signal or molecule (for example, a transcription factor). Both constitutive (e.g., GPD or CYC1 promoter) or inducible (e.g., GAL1 or CUP1 promoter) promoters can be used in the fungi and the compositions and methods provided herein. In addition, native or non-native promoters (e.g., endogenous or exogenous) can be used to drive expression of a nucleic acid of interest.

Exemplary promoters that can be used include, but are not limited to: GPD promoter, GAL1 promoter, CUP1 promoter, ADHI promoter, CYC1 promoter, PGK1 promoter, SV40 promoter, CMV enhancer-promoter, CMV enhancer/β-actin promoter, and trpC promoter. In some aspects, the promoter is a Saccharomyces cerevisiae promoter. Promoters produced by recombinant DNA or synthetic techniques are also known and useful for compositions and methods described herein (see, e.g., Feng and Marchisio, “Saccharomyces cerevisiae Promoter Engineering before and during the Synthetic Biology Era,” Biology (Basel) 10(6):504, 2021). In some aspects, the promoter is a viral promoter, for example a totivirus promoter.

Recombinant: A nucleic acid or amino acid sequence that is not naturally occurring. Recombinant sequences include, for example, sequences that include one or more substitutions, deletions, or insertions, and/or sequences made by the artificial combination of two otherwise separated segments of sequence (e.g., heterologous sequences). Artificial combination can be accomplished, for example, by chemical synthesis or artificial manipulation of isolated segments of nucleic acids, for example, through genetic engineering techniques. In several aspects, a recombinant protein is encoded by a recombinant nucleic acid molecule (e.g., a heterologous nucleic acid) that has been introduced into a host cell (e.g., a yeast cell).

Resistant: A yeast strain that is resistant to a toxin is able to grow in the presence of the toxin.

Sequence Identity: The degree of similarity between nucleic acid or amino acid sequences. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; Pearson et al., Meth. Mol. Bio. 24:307-31, 1994; and Altschul et al., J. Mol. Biol. 215:403-10, 1990. Online tools for determining sequence identity are available to the public, for example, at the NCBI website (ncbi.nlm.nih.gov) or other websites on the internet.

Unless otherwise indicated, sequence identity refers to identity over the full length of the reference sequence when optimally aligned.

Transformation: Introducing a heterologous nucleic acid into a host cell by a molecular biology technique. As used herein, the term transformation encompasses all techniques by which a nucleic acid molecule might be introduced into such a cell, including chemical methods (e.g., calcium-phosphate transfection), physical methods (e.g., electroporation, microinjection, particle bombardment), mating (e.g., rare-mating), cytoduction, fusion (e.g., liposomes), lipofection, nucleofection, receptor-mediated endocytosis (e.g., DNA-protein complexes, viral envelope/capsid-DNA complexes), biolistics (particle gun accelerator or gene gun), or other transduction and/or transfection methods.

Vector: A nucleic acid vehicle into which heterologous nucleic acids (e.g., a nucleic acid encoding K2v) can be introduced. Vectors are often used to deliver heterologous nucleic acids into host cells, for example, for replication, expression, or analysis. Vectors can include nucleic acid sequences that permit replication in the host cell, such as an origin of replication. Vectors may also include one or more antibiotic resistance genes, selectable markers, promoters, terminators, or other genetic elements.

A vector can transduce, transform or infect a cell, thereby causing the cell to express nucleic acids and/or proteins other than those native to the cell. A vector optionally includes components to aid in achieving entry of the nucleic acid into a host cell, such as a viral particle, liposome, protein coating or the like. Exemplary vectors include plasmids, viral vectors, cosmids, and artificial chromosomes. In some aspects, the vector is a plasmid (e.g., a shuttle plasmid that replicates and is expressed in both bacteria and yeast). In some aspects, the vector is a viral vector for example, a totivirus vector.

Wort: A sugary liquid extracted from grain in the process of making beer.

### III. Yeasts Including a K2v Toxin

Disclosed are engineered yeasts that include an exogenous or heterologous nucleic acid molecule encoding a K2v toxin. In some aspects, the exogenous or heterologous nucleic acid molecule is a recombinant nucleic acid molecule. The K2v toxin is a variant of the K2 toxin, which has a broader spectrum of antifungal activity relative to K2. K2v toxin, as used herein, is not or otherwise excludes canonical K2 toxin (e.g., SEQ ID NO: 5). An exemplary K2v toxin is provided herein as SEQ ID NO: 1.

The K2v toxin includes at least one mutation relative to canonical K2 toxin (e.g., SEQ ID NO: 4). In some aspects, the K2v toxin includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259, wherein the amino acid positions are according to a reference sequence set forth as either SEQ ID NO: 1 or SEQ ID NO: 4.

In some aspects, the K2v toxin includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259, wherein the amino acid positions are according to a reference sequence set forth as either SEQ ID NO: 1 or SEQ ID NO: 4.

In some aspects, the K2v toxin includes at least 80% sequence identity to SEQ ID NO: 1, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 1. In some aspects, the K2v toxin includes at least 90% sequence identity to SEQ ID NO: 1. In some aspects, the K2v toxin includes at least 95% sequence identity to SEQ ID NO: 1. In some aspects, the K2v toxin includes or consists of SEQ ID NO: 1, or a conservative variant thereof. In some aspects, the K2v toxin consists of SEQ ID NO: 1 or a conservative variant thereof.

In some aspects, the K2v toxin includes at least 90% (e.g., at least 91%, 92%, 93%, 94%, 95%, 96%, 97%, 98%, 99%, or 100%) sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259. In some aspects, the K2v toxin includes at least 90% (e.g., at least 91%, 92%, 93%, 94%, 95%, 96%, 97%, 98%, 99%, or 100%) sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259.

In some aspects, the K2v toxin includes at least 95% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259. In some aspects, the K2v toxin includes at least 95% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259, wherein the amino acid positions are according to a reference sequence set forth as SEQ ID NO: 1.

In some aspects, the K2v toxin includes at least 98% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259. In some aspects, the K2v toxin includes at least 98% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259 wherein the amino acid positions are according to a reference sequence set forth as SEQ ID NO: 1.

In some aspects, the nucleic acid molecule encoding the K2v toxin includes at least 80% sequence identity to SEQ ID NO: 2, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 2. In some aspects, the nucleic acid molecule encoding the K2v toxin includes at least 90% sequence identity to SEQ ID NO: 2. In some aspects, the nucleic acid molecule encoding the K2v toxin includes at least 95% sequence identity to SEQ ID NO: 2 and/or the nucleic acid encodes a degenerate variant of SEQ ID NO: 2. In some aspects, the nucleic acid molecule encoding the K2v toxin includes or consists of SEQ ID NO: 2 or a degenerate variant of SEQ ID NO: 2.

A nucleic acid molecule disclosed herein (e.g., a heterologous nucleic acid molecule encoding the K2v toxin) can be operably linked to one or more expression control sequences. Expression control sequences include, but are not limited to, suitable promoters, enhancers, ribosomal binding sites, transcription terminators, transcriptional regulators (e.g., AraC and LacI), start codons (e.g., ATG), splicing signals, insertions to maintain the correct reading frame, and/or stop codons.

A nucleic acid molecule disclosed herein (e.g., a heterologous nucleic acid molecule encoding the K2v toxin) can include a gene marker or tag (e.g., an antibiotic resistance marker, auxotrophic marker, protein expression marker, fluorescent tag, colorimetric tag, etc.). Selectable markers (e.g., antibiotic resistance markers or auxotrophic markers) permit selection of successful transformants based on selective agents that can be included or omitted in growth media (e.g., antibiotics or nutrients). Suitable antibiotic resistance markers include, but are not limited to, neomycin, bleomycin, or tetracycline. Suitable auxotrophic markers include, but are not limited to, URA3, HIS3, LEU2, or TRP1. Markers for protein expression can allow for screening for K2v expression by cells or clonal colonies, and include, for example fluorescent tags (e.g., GFP or RFP), luciferase, β-glucuronidase, or β-galactosidase. Tags (e.g., HA) can help facilitate protein purification and/or detection. In some aspects, a nucleic acid molecule disclosed herein (e.g., a heterologous nucleic acid molecule encoding the K2v toxin) is transcriptionally or translationally fused to a marker or tag.

The nucleic acid molecule encoding the K2v toxin can be comprised in a vector, for example, a plasmid or viral vector, or may exist as a separate molecule independent of other sequences (e.g., cDNA). Nucleotides of the nucleic acid molecule can include, for example, ribonucleotides, deoxyribonucleotides, or modified forms of either. The term “nucleic acid” includes single and double stranded forms of nucleic acids, including double stranded RNA (dsRNA). Although exemplary sequences are provided herein as DNA, it is understood that “T” is replaced by “U” in RNA molecules. In some aspects, the nucleic acid molecule is DNA, RNA, cDNA, or a synthetic nucleic acid (e.g., xenonucleic acids (XNAs)).

In several aspects, the exogenous or heterologous nucleic acid molecule encoding the K2v toxin is comprised on a vector. There are many suitable options for vectors, including for example, plasmids, viral vectors, cosmids, or artificial chromosomes (e.g. yeast). A person of ordinary skill in the art can readily identify a suitable vector for a desired application. In some aspects, the vector is a plasmid. Many plasmid vectors compatible with yeast (e.g., Saccharomyces cerevisiae) are known, including cloning vectors (e.g., Gateway® cloning vectors, e.g., pCR8/GW/TOPO) and/or expression vectors (e.g., pAG426 or pAG306). In some aspects, the plasmid vector is a shuttle vector (e.g., a plasmid that has necessary elements for replication and expression in both bacteria and yeast). In some aspects, the vector is plasmid vector pAG426 or pAG306.

In a non-limiting example, an engineered yeast disclosed herein includes an exogenous or heterologous nucleic acid molecule having at least 80% sequence identity to SEQ ID NO: 3, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 3. In some aspects, the exogenous or heterologous nucleic acid molecule includes at least 90% sequence identity to SEQ ID NO: 3. In some aspects, the exogenous or heterologous nucleic acid molecule includes at least 95% sequence identity to SEQ ID NO: 3 and/or the nucleic acid molecule encodes a degenerate variant of SEQ ID NO: 3. In some aspects, the exogenous or heterologous nucleic acid molecule includes or consists of SEQ ID NO: 3 or a degenerate variant thereof.

In a non-limiting example, an engineered yeast disclosed herein includes an exogenous or heterologous nucleic acid molecule having at least 80% sequence identity to SEQ ID NO: 18, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 18. In some aspects, the exogenous or heterologous nucleic acid molecule includes at least 90% sequence identity to SEQ ID NO: 18. In some aspects, the exogenous or heterologous nucleic acid molecule includes at least 95% sequence identity to SEQ ID NO: 18 and/or the nucleic acid molecule encodes a degenerate variant of SEQ ID NO: 18. In some aspects, the exogenous or heterologous nucleic acid molecule includes or consists of SEQ ID NO: 18, or a degenerate variant thereof.

In some aspects, the vector is a viral vector. In some aspects, the viral vector is a virus-like particle (VLP), for example, M2 satellite virus. M2 satellite virus is a small, dsRNA VLP that requires the presence of a helper virus in order to replicate (non-autonomous). Viruses of the family Totiviridae can act as helper viruses for M2 satellite virus by replicating and encapsidating the M2 satellite virus. In some aspects, a yeast including a M2 satellite virus encoding K2v further includes a totivirus competent to replicate the M2 satellite virus. In some aspects, the M2 satellite virus and/or totivirus are introduced into engineered yeast by cytoduction (see, e.g., Young et al., J I Brewing 87:292-295).

In some aspects, the nucleic acid molecule encoding the K2v toxin exists as a cytoplasmic nucleic acid (e.g., double stranded RNA) in the engineered yeast. In some aspects, the nucleic acid molecule (e.g., DNA) encoding the K2v toxin is incorporated into genomic DNA of the engineered yeast.

In several aspects, an engineered yeast disclosed herein is a brewer yeast, for example, Saccharomyces cerevisiae. The yeast can be a particular strain of Saccharomyces cerevisiae, for example, a strain used to brew beer. In some aspects, the yeast is Saccharomyces cerevisiae strain OYL-019, OYL-025, OYL-026, OYL-027, OYL-039, OYL-040, OYL-042, OYL-055, OYL-056, OYL-112, OYL-200, OYL-205, OYL-500, OYL-501, TVM, Belle Saison, AAB, AAQ, AAR, AEQ, AFA, AFB, AFP, APP, AQG, AQH, ASB, BRM, CBN, CFF, CPD, SACE_YAB, SACE_YAG, SACE_YDD, VIC-23, WLP-001, BY4741, CHB, SACE_YCA, BLG, ACP, CHD, BSG, CYC1058, CYC1172, DMS 70454, YSM1307, OS179, OS40, MS300C, or OS294. In some aspects, the yeast is Saccharomyces cerevisiae strain OYL090 (Omega Yeast San Diego Super), WLP001 (White Laps California Ale Yeast), CBC1 (Lallemand conditioning yeast), WLP028 (White Labs Edinburgh Scottish Ale), WLP940 (White Labs Mexican Lager Yeast), L28 (Imperial Yeast Urkel Czech lager yeast), OYL061 (Omega Yeast Voss Kveik), or OYL011. In some aspects, the yeast is Saccharomyces cerevisiae strain OYL-019, OYL-56, OYL-25, or OYL-112.

Also encompassed by the disclosure are methods of making engineered yeasts that include an exogenous or heterologous nucleic acid molecule encoding a K2v toxin disclosed herein, such methods include introducing the exogenous or heterologous nucleic acid molecule into a yeast cell (e.g., S. cerevisiae), thereby producing the engineered yeast. Introducing the exogenous or heterologous nucleic acid molecule can be achieved using any suitable transformation method, for example and without limitation, chemical methods (e.g., lithium chloride transformation), physical methods (e.g., electroporation, microinjection, particle bombardment), mating methods, cytoduction, fusion (e.g., liposomes), lipofection, nucleofection, receptor-mediated endocytosis (e.g., DNA-protein complexes, viral envelope/capsid-DNA complexes), biolistics (particle gun accelerator or gene gun), or other transduction and/or transfection method.

In some aspects, a gene editing system, for example, CRISPR-based gene/genome editing technology, is used to introduce the exogenous or heterologous nucleic acid molecule into a yeast, thereby making an engineered yeast. CRISPR (Clustered Regularly Interspaced Short Palindromic Repeats) technology, including systems that use, for example, Cas9 (e.g., SpCas9, SaCas9, etc.), Cas12a, Cas12i (e.g., hfCas12Max), Cas3, CasX (PlmCas12e), or Cas13, have been described, for example, in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, 8,906,616, and Koonin et al. “Discovery of Diverse CRISPR-Cas Systems and Expansion of the Genome Engineering Toolbox.” Biochemistry. doi: 10.1021/acs.biochem.3c00159, 2023. A person of ordinary skill in the art could readily select an appropriate CRISPR/Cas system for the desired application.

In a non-limiting example, a CRISPR/Cas9 system is used to introduce the exogenous or heterologous nucleic acid molecule into the genome of yeast, thereby producing an engineered yeast. In some aspects, a CRISPR/Cas9 system is used to induce a double stranded break in a DNA target sequence and the exogenous or heterologous nucleic acid molecule is provided in the form of a repair template, which is then incorporated at the site of the double-stranded break. Known methods of CRISPR/Cas9 genome editing are useful for the compositions and methods disclosed herein. A non-limiting example is provided in Ryan et al., “CRISPR-Cas9 Genome Engineering in Saccharomyces cerevisiae Cells,” Cold Spring Harb. Protoc, 2016(6), 2016.

In several aspects, a transformed nucleic acid encoding K2v includes a tag or selectable marker. In such examples, transformed yeast can be grown on selective media or screened for successful transformation. Successful transformants can be passaged one or more times on suitable media to isolate clones including the exogenous or heterologous nucleic acid molecule encoding K2v.

Expression of an exogenous or heterologous nucleic acid molecule encoding K2v can be confirmed by detecting the production of K2v transcripts or K2v protein by transformed yeast. Detection of nucleic acids or protein can be performed using known methods, for example, PCR, probe-based assays (e.g., microarray or northern blot), sequencing, ELISA, Western blot, or mass spectrometry. Detection can also be accomplished, for example, by detecting a tag (e.g., fluorescent tag (e.g., GFP or RFP), luciferase, β-glucuronidase, or β-galactosidase). In several aspects, transformed yeast are maintained in suitable cell culture or frozen until use.

### IV. Methods of Inhibiting Diastatic Yeast Growth

Also disclosed are methods of inhibiting diastatic yeast growth in a fermented beverage product, including inoculating the product, or a pre-fermented form thereof (e.g., malt, mash, or wort), with an engineered yeast including an exogenous or heterologous nucleic acid molecule encoding a K2-variant (K2v) toxin (e.g., SEQ ID NO: 1). In some aspects, the method includes inoculating the product, or a pre-fermented form thereof (e.g., malt, mash, or wort), with an engineered yeast disclosed herein. In some aspects, the exogenous or heterologous nucleic acid molecule encoding the K2v toxin is an exogenous or heterologous nucleic acid disclosed herein (e.g., SEQ ID NO: 1).

The K2v toxin includes at least one mutation relative to canonical K2 toxin (e.g., SEQ ID NO: 4). In some aspects, the K2v toxin includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259, wherein the amino acid positions are according to a reference sequence set forth as either SEQ ID NO: 1 or SEQ ID NO: 4.

In some aspects, the K2v toxin includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259, wherein the amino acid positions are according to a reference sequence set forth as either SEQ ID NO: 1 or SEQ ID NO: 4.

In some aspects, the K2v toxin includes at least 80% sequence identity to SEQ ID NO: 1, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 1. In some aspects, the K2v toxin includes at least 90% sequence identity to SEQ ID NO: 1. In some aspects, the K2v toxin includes at least 95% sequence identity to SEQ ID NO: 1. In some aspects, the K2v toxin includes or consists of SEQ ID NO: 1.

In some aspects, the K2v toxin includes at least 90% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259. In some aspects, the K2v toxin includes at least 90% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259.

In some aspects, the K2v toxin includes at least 95% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259. In some aspects, the K2v toxin includes at least 95% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259.

In some aspects, the K2v toxin includes at least 98% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and/or a threonine (T) at amino acid position 259. In some aspects, the K2v toxin includes at least 98% sequence identity to SEQ ID NO: 1 and further includes a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259.

In some aspects, the exogenous or heterologous nucleic acid molecule encoding K2v includes at least 80% sequence identity to SEQ ID NO: 2, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 2. In some aspects, the exogenous or heterologous nucleic acid molecule encoding K2v includes at least 90% sequence identity to SEQ ID NO: 2. In some aspects, the exogenous or heterologous nucleic acid molecule encoding K2v includes at least 95% sequence identity to SEQ ID NO: 2. In some aspects, the exogenous or heterologous nucleic acid molecule encoding K2v includes or consists of SEQ ID NO: 2.

In some aspects, the nucleic acid molecule encoding the K2v toxin exists as a cytoplasmic nucleic acid (e.g., double stranded RNA) in the yeast. In some aspects, the nucleic acid molecule (e.g., DNA) encoding the K2v toxin is incorporated into genomic DNA of the yeast.

The exogenous or heterologous nucleic acid molecule can be included on a vector, for example, a plasmid or viral vector. In some aspects, the vector is a viral vector, for example, a virus-like particle (VLP) (e.g., M2 satellite virus). In some aspects, a yeast including a M2 satellite virus encoding K2v further includes a totivirus competent to replicate the M2 satellite virus. In some aspects, the M2 satellite virus and/or totivirus are introduced into the engineered yeast by cytoduction (see, e.g., Young et al., J I Brewing 87:292-295).

In some aspects, the exogenous or heterologous nucleic acid molecule is included on a plasmid vector, for example, pAG426 or pAG306. In a non-limiting example, an engineered yeast disclosed herein includes an exogenous or heterologous nucleic acid molecule having at least 80% sequence identity to SEQ ID NO: 3, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 3. In some aspects, the exogenous or heterologous nucleic acid molecule includes at least 90% sequence identity to SEQ ID NO: 3. In some aspects, the exogenous or heterologous nucleic acid molecule includes at least 95% sequence identity to SEQ ID NO: 3 and/or the exogenous or heterologous nucleic acid molecule encodes a degenerate variant of SEQ ID NO: 3. In some aspects, the exogenous or heterologous nucleic acid molecule includes or consists of SEQ ID NO: 3, or a degenerate variant thereof.

In some aspects, an engineered yeast disclosed herein includes an exogenous or heterologous nucleic acid molecule having at least 80% sequence identity to SEQ ID NO: 18, for example, at least 85%, at least 88%, at least 90%, at least 91%, at least 92%, at least 93%, at least 94%, at least 95%, at least 96%, at least 97%, at least 98%, at least 99%, or 100% sequence identity to SEQ ID NO: 18. In some aspects, the exogenous or heterologous nucleic acid molecule includes at least 90% sequence identity to SEQ ID NO: 18. In some aspects, the exogenous or heterologous nucleic acid molecule includes at least 95% sequence identity to SEQ ID NO: 18 and/or the exogenous or heterologous nucleic acid molecule encodes a degenerate variant of SEQ ID NO: 18. In some aspects, the exogenous or heterologous nucleic acid molecule includes or consists of SEQ ID NO: 18, or a degenerate variant thereof.

The engineered yeast can be any yeast suitable for fermentation of a beverage product for human consumption. In some aspects, the engineer yeast is a brewer's yeast, for example, Saccharomyces cerevisiae. The yeast can be a particular strain of Saccharomyces cerevisiae, for example, a strain used to brew beer. In some aspects, the yeast is Saccharomyces cerevisiae strain OYL-019, OYL-025, OYL-026, OYL-027, OYL-039, OYL-040, OYL-042, OYL-055, OYL-056, OYL-112, OYL-200, OYL-205, OYL-500, OYL-501, TVM (Omega Yeast®), Belle Saison (Renaissance Yeast®), AAB, AAQ, AAR, AEQ, AFA, AFB, AFP, APP, AQG, AQH, ASB, BRM, CBN, CFF, CPD, SACE_YAB, SACE_YAG, SACE_YDD, VIC-23 (Vivace®, Renaissance Yeast®), WLP001, BY4741 (see, e.g., ATCC 4040002), CHB, SACE_YCA, BLG, ACP, CHD, BSG, CYC1058, CYC1172, DMS 70454, YSM1307, OS179, OS40, MS300C, or OS294. In some aspects, the yeast is Saccharomyces cerevisiae strain OYL090 (Omega Yeast® San Diego Super), WLP001 (White Laps California Ale Yeast), CBC1 (Lallemand conditioning yeast), WLP028 (White Labs Edinburgh Scottish Ale), WLP940 (White Labs Mexican Lager Yeast), L28 (Imperial Yeast Urkel Czech lager yeast), OYL061 (Omega Yeast® Voss Kveik), or OYL011 (British Ale V).

Diastatic yeast are strains of Saccharomyces cerevisiae that produce an extracellular glucoamylase encoded by a STA gene (e.g. STA1, STA2, and/or STA3). In some aspects, the diastatic yeast produces an extracellular glucoamylase encoded by STA1. In some aspects, the diastatic yeast include one or more STA+ yeast strains provided herein in Table 1.

In some aspects, the methods include a step of detecting the presence of diastatic yeast in the fermented beverage product or pre-fermented form thereof. In some aspects, diastatic yeast are detected by detecting a STA gene or protein product (e.g., STA1). A STA gene can be detected, for example, by a PCR method (e.g., amplification of a target gene), hybridization method (e.g., probe-based detection), or sequencing method (e.g., sanger sequencing, high-throughput sequencing, or pyrosequencing).

Inoculation of the engineered yeast can occur before or after fermentation of the fermented beverage product. When inoculation occurs before fermentation, the pre-fermented beverage product (e.g., wort) is inoculated with the engineered yeast and fermentation occurs in the presence of the engineered yeast. In some aspects, inoculation prior to fermentation inhibits or prevents the growth of diastatic yeast throughout the brewing process. When inoculation occurs after fermentation, the engineered yeast can be added to prevent and/or remediate diastatic yeast growth. In a non-limiting example, diastatic yeast contamination is suspected or detected in a fermented beverage product and the engineered yeast is added to the fermented beverage product, thereby eliminating the contamination.

In a non-limiting example, diastatic yeast contamination is suspected or detected in a fermented beverage product and the engineered yeast is added to the fermented beverage product, thereby eliminating the contamination. In some aspects, the engineered yeast is added to the fermented beverage product as a preventative measure to reduce risk of diastatic yeast contamination. In some aspects, the engineered yeast is added to the fermented beverage product as a remedial measure to reduce or eliminate diastatic yeast contamination.

In some aspects, toxin sensitivity is assayed prior to inoculation to determine whether contaminating diastatic yeast are resistant to K1, K2, and/or K2v toxin. In some aspects, when diastatic yeast resistant to K1 and/or K2 toxin are detected, the fermented beverage product (or pre-fermented form thereof) is inoculated with the engineered yeast including the exogenous or heterologous nucleic acid molecule encoding the K2v toxin. In some aspects, when diastatic yeast resistant to K2v toxin are detected, the fermented beverage product (or pre-fermented form thereof) is not inoculated with the engineered yeast including the exogenous or heterologous nucleic acid molecule encoding the K2v toxin. In some aspects, toxin sensitivity assays are performed in culture (e.g., agar plates).

A diastatic yeast that is resistant to a toxin (e.g., K1, K2, and/or K2v) is a diastatic yeast that continues to grow in the presence of the toxin. 100% resistance is not required. In some aspects, a resistant diastatic yeast maintains at least 50% (e.g., 60%, 70%, 75%, 80%, 85%, 90%, 95%, 98%, or 100% etc.) of its growth in the presence of a toxin relative to a control (e.g., growth under the same conditions without the toxin). In some aspects, a resistant diastatic yeast maintains at least 75% of its growth in the presence of a toxin relative to a control. In some aspects, a resistant diastatic yeast maintains at least 90% of its growth in the presence of a toxin relative to a control.

In some aspects, the growth of a resistant diastatic yeast is inhibited by 50% or less (e.g., 40%, 45%, 30%, 35%, 25%, 20%, 15%, 10%, 5%, etc.) in the presence of a toxin relative to a control (e.g., growth in the absence of the toxin). In some aspects, the growth of a resistant diastatic yeast is inhibited by 25% or less in the presence of a toxin relative to a control. In some aspects, the growth of a resistant diastatic yeast is inhibited by 10% or less in the presence of a toxin relative to a control. In some aspects, the growth of a resistant diastatic yeast is not inhibited in the presence of a toxin relative to a control.

The fermented beverage product can include, but is not limited to, beer, wine, cider, and kombucha. In some aspects, the fermented beverage product does not include wine. In some aspects, the fermented beverage product does not include wine, cider, or kombucha. In some aspects, the fermented beverage product is beer. In some aspects, the beer is a low-gravity beer (e.g., pilsner, kolsch, American lager, pale ale, English mild ale, session beer, Wiess beer, etc.). In some aspects, the beer is beer that is not produced using STA1 positive yeasts. In some aspects, the beer is not a high-gravity beer (e.g., Belgian-style beer, Brut IPA, Saison, etc.).

Brewing beer is a multi-step process that typically includes malting, milling, mashing, extract separation, hop addition and boiling, removal of hops and precipitates, cooling and aeration, pitching yeast, then fermentation. While many suitable methods of brewing beer are known, the general process is described herein. Grains (malted or unmalted) are milled and soaked in hot water, creating the mash. Most, but not all, brewing grains are malted before use. During mashing, starches from the grains are converted to fermentable sugars. The aqueous portion is separated from the spent grain and is boiled. Hops are typically added during the boiling stage. After boiling more hops or spices can be added, and any solids that have formed can be removed for a more desirable consistency. The resulting liquid composition is referred to as the wort. After the wort cools, yeast is added for fermentation. The product is typically fermented until a desired gravity is reached.

In some aspects, the methods disclosed herein inhibit diastatic yeast growth in beer. In a non-limiting example, the engineered yeast is added to wort when a beer is brewed, for example, to prevent or inhibit diastatic yeast growth. In other non-limiting examples, the engineered yeast is added to the beer after it is brewed, for example, to remediate suspected or detected diastatic yeast contamination.

In some aspects, the wort has an original gravity of 0.5° P to 2.0° P, for example, 0.8° P to 2.0° P, 1.0° P to 1.6° P, or 1.04° P to 1.06° P. In some aspects, the wort has an original gravity of less than 1.075° P. In some aspects, the wort has an original gravity of less than 1.0° P.

The methods disclosed herein can include periodic screening for the presence of diastatic yeast to ensure that the product does not contain viable diastatic yeast. Such screens can include, for example, PCR-based detection of nucleic acids specific to diastatic yeast (e.g., STA gene).

The diastatic yeast can be resistant to canonical killer toxins, such as K1 and/or K2 toxin. In some aspects, the diastatic yeast is resistant to canonical K2 toxin. In some aspects, the methods include selecting a fermented beverage product that is contaminated, or suspected of being contaminated, with diastatic yeast that are resistant to canonical K2 toxin. The methods can include a step of screening for the presence of diastatic yeast resistant to canonical K2 toxin.

Challenges with using K2 killer toxins to control diastatic yeast in the fermentation process includes unintentional changes to properties of the fermented beverage product, for example, changes in stability or final gravity, or the introduction of off-flavors, for example and without limitation, a noticeable and undesirable phenolic flavor. In some aspects, the methods disclosed herein do not negatively affect the stability (e.g., shelf life) and/or final gravity of the fermented beverage product. In some aspects, the methods disclosed herein do not negatively affect flavor of the fermented beverage product. In a non-limiting example, the methods disclosed herein do not significantly or perceivably change the flavor of a fermented beverage product relative to the fermented beverage product produced absent the engineered yeast. Flavor can be assessed, for example, by a taste testing panel. Flavor can also be assessed, for example, by comparing a fermented beverage product including the engineered yeast with a control fermented beverage product that does not include the engineered yeast.

In some aspects, the methods disclosed herein do not significantly or perceivably change the gravity (and alcohol by volume (ABV)), turbidity, color, pH, carbonation, and/or bitterness units (IBU) of the fermented beverage product relative to the fermented beverage product produced absent the engineered yeast.

### V. Donor Yeasts and Methods of Use Thereof

Further provided is Saccharomyces cerevisiae strain designated JM001, wherein a representative sample of cells of Saccharomyces cerevisiae strain JM001 has been deposited under ATCC Accession No. PTA-______. Strain JM001 is a donor yeast strain capable of accepting and transferring cytoplasmic double-stranded RNA (dsRNA) from other yeast strains. In some aspects, JM001 includes cytoplasmic dsRNA. In other aspects, JM001 does not include cytoplasmic dsRNA. JM001 is useful, for example, for moving dsRNA satellites encoding a yeast toxin (e.g., K1, K1L, K2, K2v, K21/K66, K28, K62, K74, or Klus) from a laboratory strain of yeast to a brewer strain of yeast.

In some aspects, strain JM001 includes cytoplasmic double-stranded RNA (dsRNA) encoding a yeast toxin. In some aspects, the yeast toxin inhibits growth of diastatic yeast. In some aspects, the yeast toxin is a K2v toxin disclosed herein.

Also provided are methods of transforming a yeast, including performing mating or cytoduction between Saccharomyces cerevisiae strain JM001 and at least one other yeast strain. In some aspects, mating or cytoduction facilitates transfer of cytoplasmic dsRNA from one yeast strain to another.

Depending on context, strain JM001 can either be a donor or acceptor of cytoplasmic double-stranded RNA (dsRNA). In some aspects, the at least one other strain of yeast includes cytoplasmic dsRNA, which is transferred to JM001 upon mating or cytoduction with the at least one other yeast strain. In some aspects, JM001 includes cytoplasmic dsRNA, which is transferred to the at least one other yeast strain upon the mating or cytoduction.

The cytoplasmic dsRNA can encode a protein, for example, a yeast toxin. In some aspects, the yeast toxin is a toxin that inhibits diastatic yeast growth (e.g., K1, K2, or Kv2). In some aspects, the yeast toxin is a toxin disclosed herein (e.g., K2v). In some aspects, Saccharomyces cerevisiae strain JM001 or the at least one other yeast strain includes a nucleic acid disclosed herein (e.g., a nucleic acid or vector encoding K2v). Toxin production by Saccharomyces yeasts is accompanied by immunity to the toxin. Thus, yeasts expressing the yeast toxin are immune to their own toxins.

The methods can include a step of selecting for or isolating transformed yeast. Selection of successful transformants can be based on selective agents that are included or omitted in growth media (e.g., antibiotics or nutrients). Suitable antibiotic resistance markers include, but are not limited to, neomycin, bleomycin, or tetracycline. Suitable auxotrophic markers include, but are not limited to, URA3, HIS3, LEU2, or TRP1. Markers of protein expression can also allow for screening for transformed cells or clonal colonies, and include, for example fluorescent tags (e.g., GFP or RFP), luciferase, β-glucuronidase, or β-galactosidase. An appropriate selective media is determined by the selective markers present in the transformants. In some aspects, the methods include a step of selecting for transformed JM001 or a transformed brewer yeast. JM001 is an uracil auxotroph and therefore can at least be selected for based on uracil auxotrophy.

In some aspects, the methods include detecting expression of a yeast toxin (e.g., K1, K2, or Kv2) by the transformed yeast (e.g., transformed JM001 or a transformed brewer yeast). In a non-limiting example, production of the yeast toxin is confirmed by observing a killer phenotype when the transformed yeast is co-cultured with a yeast that is susceptible to the yeast toxin. In some aspects, the transformed yeast is co-cultured with the susceptible yeast on YPD media including methylene blue (pH 4.6).

Strain JM001 can also be used in methods of producing an engineered yeast disclosed herein. In such methods, JM001 is used as a vehicle to move dsRNA encoding the K2v toxin into a second yeast strain, for example, a brewer yeast strain, thereby producing the engineered yeast. In some aspects, the dsRNA is engineered to encode the K2v toxin (the dsRNA is not a naturally occurring molecule).

In some aspects, the brewer yeast is a strain of Saccharomyces cerevisiae used to brew beer. In some aspects, the brewer yeast is Saccharomyces cerevisiae strain OYL-019, OYL-025, OYL-026, OYL-027, OYL-039, OYL-040, OYL-042, OYL-055, OYL-056, OYL-112, OYL-200, OYL-205, OYL-500, OYL-501, TVM, Belle Saison, AAB, AAQ, AAR, AEQ, AFA, AFB, AFP, APP, AQG, AQH, ASB, BRM, CBN, CFF, CPD, SACE_YAB, SACE_YAG, SACE_YDD, VIC-23, WLP-001, BY4741, CHB, SACE_YCA, BLG, ACP, CHD, BSG, CYC1058, CYC1172, DMS 70454, YSM1307, OS179, OS40, MS300C, or OS294.

Suitable methods of cytoduction or mating (e.g., rare-mating) have been described, and a practitioner can select an appropriate method based on the yeast strains that are used. In a non-limiting example, JM001 is rare-mated with a yeast including cytoplasmic dsRNA encoding a yeast toxin, with selection for uracil auxotrophs (to select for the donor strain genotype). Successful cytoduction is confirmed by the growth of auxotrophic yeast on medium containing glycerol as the sole carbon source. In some aspects, production of the yeast toxin is confirmed by observing a killer phenotype on YPD media (pH 4.6 with methylene blue).

### CLAUSES

Clause 1. A method of inhibiting diastatic yeast growth in a fermented beverage product, comprising:
inoculating the product or a pre-fermented form thereof with an engineered yeast comprising a heterologous nucleic acid molecule encoding a variant K2 (K2v) toxin, wherein the K2v toxin comprises an amino acid sequence having at least 90% sequence identity to SEQ ID NO: 1, and
wherein the K2v toxin comprises a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259, wherein the amino acid positions are according to a reference K2v sequence set forth as SEQ ID NO: 1.

Clause 2. The method of clause 1, wherein the fermented beverage product is beer.

Clause 3. The method of any one of the prior clauses, wherein inoculation with the engineered yeast occurs prior to fermentation.

Clause 4. The method of any one of the prior clauses, wherein the K2v toxin comprises:
(i) at least 95% sequence identity to SEQ ID NO: 1;
(ii) at least 98% sequence identity to SEQ ID NO: 1; and/or
(iii) at least 99% sequence identity to SEQ ID NO: 1.

Clause 5. The method of any one of the prior clauses, wherein the K2v toxin comprises or consists of SEQ ID NO: 1.

Clause 6. The method of any one of the prior clauses, wherein the heterologous nucleic acid molecule comprises:
(i) at least 90% sequence identity to SEQ ID NO: 2;
(ii) at least 95% sequence identity to SEQ ID NO: 2; and/or
(iii) at least 98% sequence identity to SEQ ID NO: 2.

Clause 7. The method of any one of the prior clauses, wherein the heterologous nucleic acid molecule comprises SEQ ID NO: 2.

Clause 8. The method of any one of the prior clauses, wherein the heterologous nucleic acid molecule is comprised on a vector; optionally wherein the vector is a viral or plasmid vector.

Clause 9. The method of any one of the prior clauses, wherein the viral vector is a M2 satellite virus vector.

Clause 10. The method of any one of the prior clauses, wherein the heterologous nucleic acid molecule is cytoplasmic double-stranded RNA (dsRNA).

Clause 11. The method of any one of clauses 1-8, wherein the vector is a plasmid.

Clause 12. The method of any one of the prior clauses, wherein the heterologous nucleic acid molecule comprises:
(i) at least 90% sequence identity to SEQ ID NO: 3 or SEQ ID NO: 18;
(ii) at least 95% sequence identity to SEQ ID NO: 3 or SEQ ID NO: 18; and/or
(iii) at least 98% sequence identity to SEQ ID NO: 3 or SEQ ID NO: 18.

Clause 13. The method of any one of the prior clauses, wherein the heterologous nucleic acid molecule comprises or consists of SEQ ID NO: 3 or SEQ ID NO: 18.

Clause 14. The method of any one of the prior clauses, wherein the engineered yeast is S. cerevisiae.

Clause 15. The method of any one of the prior clauses, wherein the engineered yeast is a brewer yeast, optionally wherein the brewer yeast is OYL090 (Omega Yeast San Diego Super), WLP001 (White Laps California Ale Yeast), CBC1 (Lallemand conditioning yeast), WLP028 (White Labs Edinburgh Scottish Ale), WLP940 (White Labs Mexican Lager Yeast), L28 (Imperial Yeast Urkel Czech lager yeast), OYL061 (Omega Yeast Voss Kveik), or OYL011.

Clause 16. The method of any one of the prior clauses, wherein the diastatic yeast is resistant to canonical K2 toxin set forth as SEQ ID NO: 4.

Clause 17. The method of any one of the prior clauses, further comprising selecting the fermented beverage product as having or suspected of having diastatic yeast contamination.

Clause 18. A method of inhibiting diastatic yeast growth in a process of making beer, comprising:
(i) selecting a wort that has or is suspected of having contamination with a diastatic yeast that is resistant to canonical K2 toxin set forth as SEQ ID NO: 4;
(ii) inoculating the wort with a S. cerevisiae expressing a variant K2 toxin as represented by SEQ ID NO: 1; and
(iii) fermenting the wort to produce beer;
thereby inhibiting diastatic yeast growth in the process of making beer.

Clause 19. The method of clause 18, wherein the beer does not have an undesirable phenolic flavor.

Clause 20. An engineered yeast comprising a recombinant vector encoding a K2v toxin, wherein:
the K2v toxin comprises an amino acid sequence having at least 90% sequence identity to SEQ ID NO: 1,
the K2v toxin comprises a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259, wherein the amino acid positions are according to a reference K2v sequence set forth as SEQ ID NO: 1, and
the recombinant vector comprises at least 90% sequence identity to SEQ ID NO: 3.

Clause 21. The engineered yeast of clause 20, wherein the K2v toxin comprises:
(i) at least 95% sequence identity to SEQ ID NO: 1;
(ii) at least 98% sequence identity to SEQ ID NO: 1; and/or
(iii) at least 99% sequence identity to SEQ ID NO: 1.

Clause 22. The engineered yeast of clause 20 or clause 21, wherein the engineered yeast is S. cerevisiae.

Clause 23. The engineered yeast of any one of clauses 20-22, wherein the engineered yeast is a brewer yeast, optionally wherein the brewer yeast is OYL090 (Omega Yeast San Diego Super), WLP001 (White Laps California Ale Yeast), CBC1 (Lallemand conditioning yeast), WLP028 (White Labs Edinburgh Scottish Ale), WLP940 (White Labs Mexican Lager Yeast), L28 (Imperial Yeast Urkel Czech lager yeast), OYL061 (Omega Yeast Voss Kveik), or OYL011.

Clause 24. The engineered yeast of any one of clauses 20-23, wherein the recombinant vector is a plasmid.

Clause 25. The engineered yeast of any one of clauses 20-24, wherein the recombinant vector comprises:
(i) at least 95% sequence identity to SEQ ID NO: 3;
(ii) at least 98% sequence identity to SEQ ID NO: 3; and/or
(iii) at least 99% sequence identity to SEQ ID NO: 3.

Clause 26. The engineered yeast of any one of clauses 20-25, wherein the recombinant vector comprises or consists of SEQ ID NO: 3.

Clause 27. The engineered yeast of any one of clauses 20-26, wherein the recombinant vector is a M2 satellite virus vector.

Clause 28. The engineered yeast of any one of clauses 20-23 or 27, wherein the recombinant vector is cytoplasmic double-stranded RNA (dsRNA).

Clause 29. A method of inhibiting diastatic yeast growth in a fermented beverage product, comprising inoculating the fermented beverage product, or a pre-fermented form thereof, with the engineered yeast of any one of clauses 20-28.

### EXAMPLES

### Example 1

### Materials and Methods

### Microbial Strains and Growth Conditions.

The names and origins of strains used in this study are provided in Table 1.

TABLE 1
Names, description, and origin of yeast strains.

| | | | Brewing | | | | |
|---|---|---|---|---|---|---|---|
| Genus | Species | Strain | nomenclature | STA1 | Killer | POF | Source |
| Saccharomyces | cerevisiae | OYL-019 | Belgian Ale D | + | nd | + | Omega |
| | | | yeast | | | | Brewing |
| Saccharomyces | cerevisiae | OYL-025 | Bavarian | + | − | + | Omega |
| | | | Wheat I yeast | | | | Brewing |
| Saccharomyces | cerevisiae | OYL-026 | French Saison | + | nd | + | Omega |
| | | | yeast | | | | Brewing |
| Saccharomyces | cerevisiae | OYL-027 | Belgian Saison | + | nd | + | Omega |
| | | | yeast | | | | Brewing |
| Saccharomyces | cerevisiae | OYL-039 | Biere de garde | + | nd | + | Omega |
| | | | yeast | | | | Brewing |
| Saccharomyces | cerevisiae | OYL-040 | Belgian Dark | + | nd | + | Omega |
| | | | Ale | | | | Brewing |
| Saccharomyces | cerevisiae | OYL-042 | Belgian Saison | + | nd | + | Omega |
| | | | II yeast | | | | Brewing |
| Saccharomyces | cerevisiae | OYL-055 | Vermont | + | nd | + | Omega |
| | | | Farmhouse Ale | | | | Brewing |
| Saccharomyces | cerevisiae | OYL-056 | Belgian golden | + | − | + | Omega |
| | | | strong yeast | | | | Brewing |
| Saccharomyces | cerevisiae | OYL-112 | Swiss Lager | + | − | + | Omega |
| | | | | | | | Brewing |
| Saccharomyces | cerevisiae | OYL-200 | Tropical IPA | + | nd | | Omega |
| | | | yeast | | | | Brewing |
| Saccharomyces | cerevisiae | OYL-205 | Tropical IPA | + | nd | − | Omega |
| | | | | | | | Brewing |
| Saccharomyces | cerevisiae | OYL-500 | Saisonstein | + | nd | + | Omega |
| | | | yeast | | | | Brewing |
| Saccharomyces | cerevisiae | OYL-501 | Gulo yeast | + | nd | − | Omega |
| | | | | | | | Brewing |
| Saccharomyces | cerevisiae | TVM | STA1 | + | nd | nd | Omega |
| | | | | | | | Brewing |
| Saccharomyces | cerevisiae | Belle | French Saison | + | nd | + | Lallamand |
| | | Saison | yeast | | | | |
| Saccharomyces | cerevisiae | AAB | N/A | + | nd | nd | Gianni |
| | | | | | | | Liti |
| Saccharomyces | cerevisiae | AAQ | N/A | + | nd | nd | Gianni |
| | | | | | | | Liti |
| Saccharomyces | cerevisiae | AAR | N/A | + | nd | nd | Gianni |
| | | | | | | | Liti |
| Saccharomyces | cerevisiae | AEQ | N/A | + | nd | nd | Gianni |
| | | | | | | | Liti |
| Saccharomyces | cerevisiae | AFA | N/A | + | − | nd | Gianni |
| | | | | | | | Liti |
| Saccharomyces | cerevisiae | AFB | N/A | + | + | nd | Gianni |
| | | | | | | | Liti |
| Saccharomyces | cerevisiae | AFP | N/A | + | − | nd | Gianni |
| | | | | | | | Liti |
| Saccharomyces | cerevisiae | APP | N/A | + | + | nd | Gianni |
| | | | | | | | Liti |
| Saccharomyces | cerevisiae | AQG | N/A | + | nd | nd | Gianni |
| | | | | | | | Liti |
| Saccharomyces | cerevisiae | AQH | N/A | + | + | nd | Gianni |
| | | | | | | | Liti |
| Saccharomyces | cerevisiae | ASB | N/A | + | − | nd | Gianni |
| | | | | | | | Liti |
| Saccharomyces | cerevisiae | BRM | N/A | + | − | nd | Gianni |
| | | | | | | | Liti |
| Saccharomyces | cerevisiae | CBN | N/A | + | nd | nd | Gianni |
| | | | | | | | Liti |
| Saccharomyces | cerevisiae | CFF | N/A | + | nd | nd | Gianni |
| | | | | | | | Liti |
| Saccharomyces | cerevisiae | CPD | N/A | + | nd | nd | Gianni |
| | | | | | | | Liti |
| Saccharomyces | cerevisiae | SACE_YAB | N/A | + | − | nd | Gianni |
| | | | | | | | Liti |
| Saccharomyces | cerevisiae | SACE_YAG | N/A | + | − | nd | Gianni |
| | | | | | | | Liti |
| Saccharomyces | cerevisiae | SACE_YDD | N/A | + | − | nd | Gianni |
| | | | | | | | Liti |
| Saccharomyces | cerevisiae | VIC-23 | Viva | − | + [M2] | − | Renaissance |
| | | | | | | | Yeast |
| Saccharomyces | cerevisiae | WLP-001 | California Ale | − | | − | White |
| | | | Yeast | | | | Labs |
| Saccharomyces | cerevisiae | BY4741 | N/A | nd | − | nd | n/a |
| Saccharomyces | cerevisiae | CHB | N/A | nd | + [M2v] | nd | Gianni |
| | | | | | | | Liti |
| Saccharomyces | cerevisiae | SACE_YCA | N/A | nd | + [M2v] | nd | Gianni |
| | | | | | | | Liti |
| Saccharomyces | cerevisiae | BLG | N/A | nd | + [M2v] | nd | Gianni |
| | | | | | | | Liti |
| Saccharomyces | cerevisiae | ACP | N/A | nd | + [M2v] | nd | Gianni |
| | | | | | | | Liti |
| Saccharomyces | cerevisiae | CHD | N/A | nd | + [M2v] | nd | Gianni |
| | | | | | | | Liti |
| Saccharomyces | cerevisiae | BSG | N/A | nd | + [M2v] | nd | Gianni |
| | | | | | | | Liti |
| Saccharomyces | cerevisiae | CYC1058 | N/A | nd | + [M2] | nd | CYC |
| Saccharomyces | cerevisiae | CYC1172 | N/A | nd | + [M2] | nd | CYC |
| Saccharomyces | cerevisiae | DMS70454 | N/A | nd | + [Mlus] | nd | DSMZ |
| Saccharomyces | cerevisiae | YSM1307 | N/A | nd | + [M1] | nd | FGSC |
| Saccharomyces | cerevisiae | OS179 | N/A | nd | + [M62] | nd | Gianni |
| | | | | | | | Liti |
| Saccharomyces | cerevisiae | OS40 | N/A | nd | + [M21] | nd | Gianni |
| | | | | | | | Liti |
| Saccharomyces | cerevisiae | MS300C | N/A | nd | + [M28] | nd | Manfred |
| | | | | | | | Schmitt |
| Saccharomyces | cerevisiae | OS294 | N/A | nd | + [M74] | N/A | Gianni |
| | | | | | | | Liti |
| Saccharomyces | paradoxus | Y63717 | N/A | nd | + [M1L] | nd | FGSC |
| Naumovozyma | dairenensis | NCYC777 | N/A | nd | + | nd | NCYC |

“+” and “−” are binary measures of the presence of a specific gene (STAI), the killer phenotype (killer), or POF (phenolic off-flavor).
“nd” indicates not done.
“N/A” indicates not applicable.

Yeasts were propagated in standard yeast extract, peptone, and dextrose medium (YPD; 10 g yeast extract, 20 g peptone, 20 g dextrose, 20 g agar in a total volume of 1 L of deionized water). Yeast strains containing killer toxin expression plasmids were maintained using complete medium (CM; 2.5 g appropriate amino acid mixture, 1.7 g yeast nitrogen base, 5 g ammonium sulfate, 20 g dextrose, 20 g agar in a total volume of 1 L of deionized water) lacking uracil with dextrose. Escherichia coli for cloning was grown using Luria broth (LB) with an appropriate antibiotic and agar (LB; 25 g LB powder, 15 g agar with a final concentration of either 10 μg mL⁻¹ of spectinomycin or 100 μg mL⁻¹ of ampicillin).

### TOPO and Gateway® Cloning of K1, K2, and K2v.

To amplify the full-length K1, K2 and K2v genes, SuperScript™ IV reverse transcriptase (18090010; Thermo®) and Phusion™ DNA Polymerase (M30530S; New England Biolabs®) were used with the primer pairs PRX542/PRUI1, PRUI115/PRUI116, and K2P1/K2P2, respectively (see, Table 2). The templates for these reactions were purified dsRNAs from S. cerevisiae strains YJM1307 (K1), ACP (K2v), and CYC1172 (K2). After cleanup with the QIAquick™ PCR purification kit, A-tails were added to the PCR products using Taq polymerase (M0273S; New England Biolabs®) following the manufacturer's recommendation. A-tailed PCR products were cloned using the pCR8/GW/TOPO® TA Cloning Kit by mixing 0.25 μL of salt solution and 0.25 μL of pCR8 vector with 1 μL of the PCR product. The solution was incubated at 25° C. for 1.5 hours. Half of the manufacturer's recommended amount of One Shot TOP10™ chemically competent E. coli (25 μL) was added to the reaction mix on ice. The mixture was then incubated on ice for 30 min, followed by 30 seconds at 42° C. and 2 minutes on ice. 250 μL of pre-warmed (37° C.) SOC was added, and the mixture was shaken at 37° C. at 220 rpm for 1 hour before being spread on LB agar plates containing spectinomycin. Plasmids were purified using the QIAprep™ Spin Miniprep Kit before analysis by restriction enzyme analysis and Sanger sequencing (using primers M13F and M13R). The insertion of K1 (YJM1307), K2v (ACP), and K2 (CYC1172) into pCR8/GW/TOPO® created the plasmids pUI101, pVZ001, and pUI099 (see, Table 3). Gateway® cloning introduced K1, K2, and K2v genes into an integrative yeast shuttle vector (pAG306-GPD-ccdB). One-quarter of the manufacturer's recommended amount of each reagent was used for each reaction: 0.5 μL of the pCR8 entry vector, 0.5 μL LR Clonase™ II enzyme mix, 0.5 μL destination plasmid and 1 μL of sterile water (Alberti et al., Yeast (Chichester, England) 24:913-919). The mixture was inoculated at 25° C. for 3 hours before adding 0.25 μL of Proteinase K and incubating at 37° C. for 10 minutes. Reaction mixtures were used to transform One Shot TOP10® chemically competent E. coli as described above but with selection by ampicillin. Plasmids were purified using the QIAprep™ Spin Miniprep Kit before being analyzed using restriction enzyme analysis. The Gateway® cloning of K2 (CYC1172) and K2v (ACP) into the high copy plasmid vector pAG426-GPD-ccdB created the plasmids pUI095 and pVZ004, respectively. The Gateway® cloning of K1 (YJM1307) and K2 (CYC1172) into the integrative vector pAG306-GPD-ccdB created the plasmids pVZ002 and pVZ003, respectively.

TABLE 2
Primer Sequences.

| Primer Name | Primer sequence |
|---|---|
| prMRK199 | TGTCGGCTAATGGTAACCTGTATGG |
| | (SEQ ID NO: 6) |
| prMRK120 | GTCACAGCCTTCAAAGTCATTATTGG |
| | (SEQ ID NO: 7) |
| prMRK123 | GTGGCCTCTTTTTATTCACCACTCC |
| | (SEQ ID NO: 8) |
| prMRK124 | GTCTCGAATCCCTCTTGACAATTCC |
| | (SEQ ID NO: 9) |
| K2P1 | ATGAAAGAGACTACCACCAGC |
| | (SEQ ID NO: 10) |
| K2P2 | GATCGGCGACAGTGTAAGTGGT |
| | (SEQ ID NO: 11) |
| PRUI115 | ATGAAAGAGACTACCACCAGCCTGATGC |
| | (SEQ ID NO: 12) |
| PRUI116 | CTAGCCGCTGTCACATTCACCATCAACC |
| | (SEQ ID NO: 13) |
| PRX542 | GAAAAATAAAGAAATGACGAAGCCAACCCAAG |
| | (SEQ ID NO: 14) |
| PRUII | GAGTTATCGCATCAGAGGTCAGACAC |
| | (SEQ ID NO: 15) |
| M13F | ACTGGCCGTCGTTTTAC |
| | (SEQ ID NO: 16) |
| M13R | GTCATAGCTGTTTCCTG |
| | (SEQ ID NO: 17) |

TABLE 3
Description of vectors.

| | | Yeast | Bacterial | |
|---|---|---|---|---|
| Name | Description | marker | marker | Reference |
| pAG306-GPD-ccdB | Gateway | URA3 | bla, cat | Alberti et al. “A suite of |
| | destination | | | gateway cloning vectors |
| | vector; | | | for high-throughput |
| | integrative | | | genetic analysis in |
| | shuttle | | | Saccharomyces |
| | vector | | | cerevisiae.” Yeast |
| | | | | 24: 913-919, 2007 |
| pCR ™8/GW/TOPO ® | TOPO-TA | n/a | aad | Commercially available |
| | cloning | | | from Thermofisher ®, see, |
| | vector and | | | e.g., catalog number |
| | Gateway | | | K250020 |
| | entry vector | | | |
| pAG426-GPD-ccdB | Gateway | URA3 | bla, cat | Alberti et al. “A suite of |
| | destination | | | gateway cloning vectors |
| | vector; high | | | for high-throughput |
| | copy | | | genetic analysis in |
| | episomal | | | Saccharomyces |
| | shuttle | | | cerevisiae.” Yeast |
| | vector | | | 24: 913-919, 2007 |
| pUI099 | pCR8 with | n/a | aad | This study |
| | K2 | | | |
| | (CYC1172) | | | |
| pVZ001 | pCR8 with | n/a | aad | This study |
| | K2v (ACP) | | | |
| pUI101 | pCR8 with | n/a | aad | This study |
| | K1 | | | |
| | (YJM1307) | | | |
| pUI095 | pAG426- | URA3 | bla | This study |
| | GPD-ccdB | | | |
| | with K2 | | | |
| pVZ004 | pAG426- | URA3 | bla | This study |
| | GPD-ccdB | | | |
| | with K2v | | | |
| pVZ002 | pAG306- | URA3 | bla | This study |
| | GPD-ccdB | | | |
| | with K1 | | | |
| pVZ003 | pAG306- | URA3 | bla | This study |
| | GPD-ccdB | | | |
| | with K2 | | | |

Curing of Satellite dsRNAs

Yeast strains to be cured of satellites were cultured in 25 mL of YPD media at 30° C. with shaking at 180 RPM to OD 1.1 mL of this culture was added to 3 mL of YPD with increasing concentration of cycloheximide (1 μM-14 μM). Cells were incubated for ~5 days at 30° C. at 180 RPM. 100 μL of these liquid cultures were spread over 10 cm YPD agar plates and incubated for 48 hours at 30° C. The resulting colonies were then examined for loss of killer toxin production.

### Double-Stranded RNA Extraction.

Double-stranded RNAs for analysis by gel electrophoresis were purified (Fredericks et al., Plos Genet 17:e1009341). Specifically, yeast cultures inoculated in yeast peptone dextrose (YPD) broth were grown overnight at 30° C. Cultures were centrifuged for 5 minutes at 8,000×g, the supernatant aspirated, and the cells washed once with sterile water. Cellulose columns were prepared by puncturing a 0.6 mL tube with a hot needle and nesting it in a 2.0 mL centrifuge tube. 0.06 g of cellulose powder D (Advantec, Japan) was added to the 0.6 mL tube, followed by 500 μL of wash buffer (1×STE (100 mM NaCl; 10 mM Tris-HCl, pH 8.0; 1 mM EDTA, pH 8.0) containing 16% (v/v) ethanol). 1×STE was added to approximately 0.04 g of wet biomass from YPD cultures and was vortexed for 3 min at 3000 rpm. 50 μL of 10% (w/v) SDS solution and 500 μL of phenol-chloroform-isoamyl alcohol [25:24:1] pH 8.0 were added to the cell suspension and vortexed until homogenous. Samples were centrifuged at 20,000×g for 5 minutes, the supernatant was transferred to a clean tube, and a second 500 μL of phenol-chloroform-isoamyl alcohol extraction was performed. The aqueous phase was transferred to a clean tube, and a one-fifth volume of ethanol was added. Tubes were mixed and centrifuged at 20,000×g for 3 minutes before the supernatant was transferred to the cellulose column and centrifuged at 10,000×g for 10 seconds. After discarding the flow-through, 400 μL of wash buffer was added to the columns, centrifuged at 10,000×g for 10 seconds, and the flow-through was discarded three times. The columns were dried by centrifugation at 10,000×g for 10 seconds. Cellulose columns were transferred to clean tubes and 400 μL of 1×STE was added, and columns were centrifuged at 10,000×g for 10 seconds to collect the eluate. 40 μL of 3 M aqueous sodium acetate (pH 5.2) and 1 mL of 100% ethanol were added to the eluate, mixed by inversion, and centrifuged at 20,000×g for 5 minutes to precipitate the dsRNAs. dsRNA pellets were allowed to air-dry before being suspended in nuclease-free water.

### Killer Toxin Assays on Agar Plates.

The general detection of killer toxin production by killer yeasts was assayed as previously described by Fredericks et al. (Fredericks et al., Plos Genet 17:e1009341) by growing yeasts on killer yeast agar plates (YPD agar plates with 0.003% w/v methylene blue buffered at pH 4.6 with sodium citrate). General screening for toxin production was identified by observing either a zone of growth inhibition or methylene blue-staining of the susceptible lawn yeasts. Specifically, approximately 6×10⁵ toxin-susceptible yeast cells were spread onto killer yeast agar plates. Cells from 2 mL overnight cultures of killer yeasts were pelleted and pinned onto susceptible lawns for qualitative assessment of killer toxin susceptibility. Approximately 6×10⁶ cells of killer toxin-expressing yeast were spotted on the inoculated plates for quantitative assays. Killer assay plates were incubated at ambient temperature for three to seven days. Killer toxin growth inhibition measurements were made after 7 days of growth using images taken by a Singer Instruments® Phenobooth+® colony counter. The images were analyzed in Fiji® (Image J®), where the colony's diameter and the inhibition area were measured. When killer toxin expression from high copy plasmids (K2v (pUI095) and K2 (pVZ004)) was induced by galactose. These strains of yeasts were maintained on complete media (CM) lacking uracil before plating onto YPD or YPG plates (yeast peptone galactose) plates.

### Detection of K1 and K2 Killer Toxin Genes

The presence of killer toxin genes on satellite dsRNAs was detected in total nucleic acid samples (prepared according to (Looke et al., BioTechniques 50:325-328) using SuperScript® IV reverse transcriptase (Thermo®) and Phusion^{T}® DNA Polymerase (New England Biolabs) as directed by the manufacturer's instructions. Primer pairs prMRK199/prMRK120 and prMRK123/prMRK124 were used to detect the canonical killer toxin genes K1 and K2, respectively. For the detection of these genes on DNA, reverse transcription was omitted. The PCR products were visualized using 1% agarose gel at 120V for 45 minutes. All primers are listed in Table 2. Genomic DNA with K1 and K2 was extracted from S. cerevisiae with integrated plasmids pVZ002 and pVZ003.

### Fermentation Trials

All yeasts were grown from pure cultures maintained cryogenically or on agar slants. Brewing yeasts for this project were propagated in four parallel flasks utilizing a stepwise 10-fold increase in volume at each step. This process started with inoculation from an agar slant into 25 mL of sterile 12° P wort into two 125 mL baffled culture flasks and placed into a shaker incubator at 28° C. This was repeated to a final volume of 5 L and a final cell count of 5.0×10⁸ cells per ml. Fermentation trials were brewed with Rahr two-row brewers' malt and 363 g of bravo hops (20 IBU). The 11.5° P wort was transferred out of the brewhouse through an inline heat exchanger to reduce wort temperature to 20° C. and inoculated with 10 L of WLP-001 at a pitching rate of 1.0×10⁶ cell/ml/° P. For both the diastatic and killer yeasts, 5×10⁴ cells mL⁻¹ were added once the gravity had stabilized for three consecutive days. The temperature during fermentation was maintained at 21° C. In trial one, 5 L of the STA1+ diastatic yeast strain Belle Saison from Lallemand was added through the hop port while CO₂ provided positive pressure. In trial two, 5 L of the same diastatic was added with 5 L of the K2 killer yeast strain Viva (VIC-23) from Renaissance Yeast through the hop port while CO₂ provided positive pressure. Cell counts were taken using an AOPI stain on the Nexcelom X2® automated cellometer. All data from both trials were collected in real time via a recirculating inline loop attached via the hop port. The instrument collected data every 30 min on pH, density (g/cm³), gravity (° P), dissolved oxygen (DO) (mg/L), conductivity (uS/cm), and temperature (° C.) using the Brew IQ® real-time data collection system. The instrumentation was cleaned with the alkaline non-caustic CIP cleaner Cell-R-Mastr®, triple rinsed with 60° C. water, and sanitized with peroxyacetic acid for 30 min before attaching to the fermenter.

### Fermentation Cellar Cooling Systems

This study's ten-hectoliter (1,000 L) pioneer fermentation vessels were jacketed and cooled via an inline re-circulating propylene glycol system. This closed-loop system employed a heat transfer fluid of propylene glycol and water that circulates to the fermenter jackets through heat exchangers. A 30RAP011 Carrier 10 Ton Glycol Chilling Unit was used to cool the 50% glycol-water mixture to 21° C. and was monitored and controlled by an Allen Bradley® human-machine interface.

### Tasting Panel

A panel of five cicerones and trained tasters were selected to participate in the off-flavor evaluation of the beer samples from both fermentation trials. Cicerones are rigorously trained to refine their olfactory and senses, enabling them to detect and differentiate specific off-flavors commonly associated with beer. Triad panels in parallel were conducted blind to determine if tasters could detect any difference between the two samples. Each beer was sampled in 118 mL pours in clear snifter glasses. Tasters were asked to rate the intensity of the selected aromas and tastes in each sample on a scale from 1-10, with 1 being absent and 10 being high.

### Example 2

### Susceptibility of Diastatic Yeast Strains to Canonical Killer Toxins

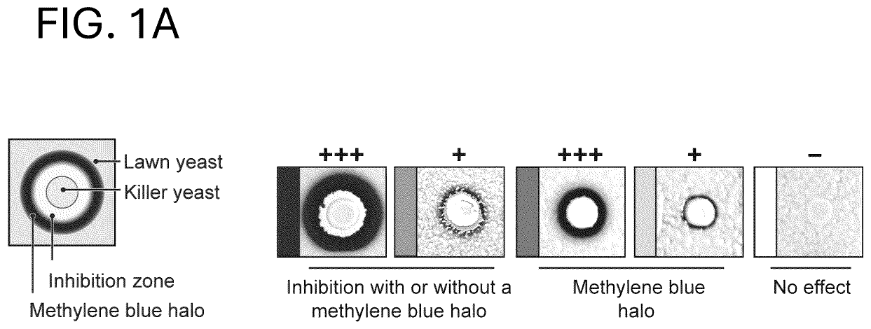

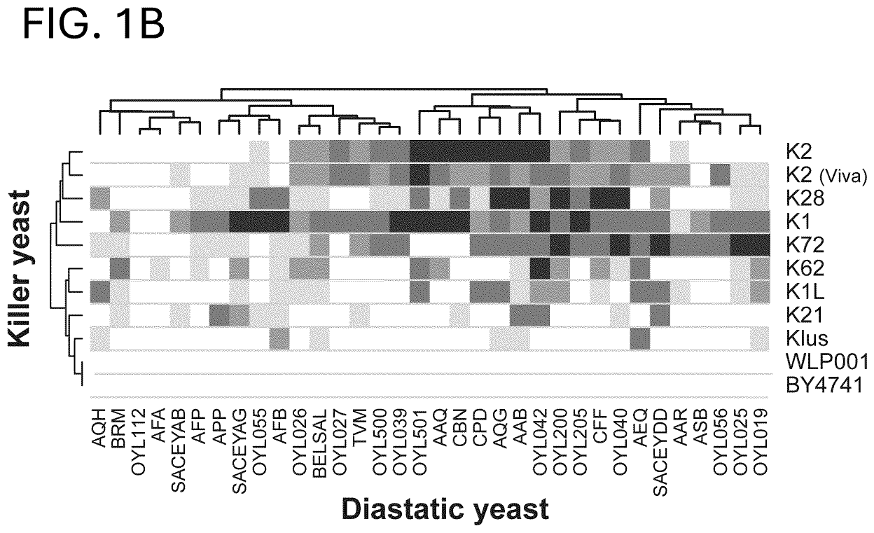

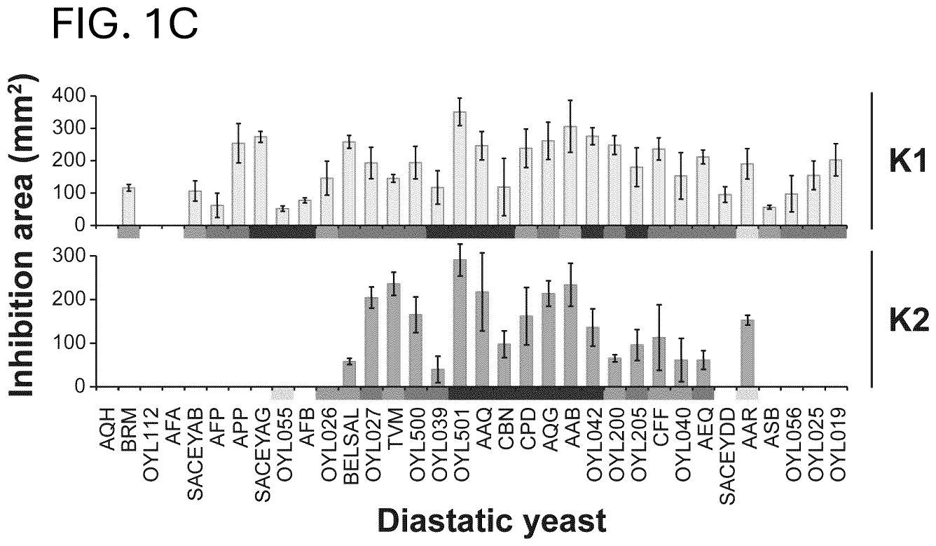

To determine the susceptibility of diastatic (STA1+) strains of S. cerevisiae to killer toxins, 34 diastatic strains were challenged by Saccharomyces yeasts expressing eight different canonical killer toxins (K1, K1L, K2, K21/K66, K28, K62, K74, and Klus) (FIGS. 1A-1B). Zones of growth inhibition and halos of methylene blue surrounding the killer yeast indicated the susceptibility of diastatic yeasts to the antifungal activities of killer toxins. Whereas zones of growth inhibition lack any observable growth of diastatic yeasts, methylene blue halos result from the initial growth of diastatic yeasts, followed by cell death due to sustained killer toxin exposure. Loss of viability results in the oxidation of methylene blue present in diastatic yeast cells and the appearance of blue-stained cells. The extent of growth inhibition was first qualitatively scored according to the degree of growth inhibition and methylene blue staining using a high throughput plating assay (FIGS. 1A-1B). Of all the canonical killer toxins assayed, K1 was judged to be the most inhibitory to diastatic yeasts and could prevent the growth of 91.2% of the diastatic strains tested. K2 could inhibit the growth of 58.8% of diastatic strains and, after K1, produced the largest zones of growth inhibition with methylene blue halos. The potency of K1 and K2 against diastatic yeast was further confirmed by quantitatively comparing the area of killer toxin inhibition against all diastatic strains tested (FIG. 1C). This analysis again found that K1 was the most effective at inhibiting diastatic yeasts. Overall, the quantitative analysis agreed with the K1 qualitative assay and had only two false positives (OYL055 and OYL026) across the K2 datasets. Overall, K1 was significantly more potent than K2, with an average area of growth inhibition of 175.5 mm² (SD; 84.5), as compared to 87.6 mm² (SD; 92.9) (Student's two-tailed T-Test, p<0.05).

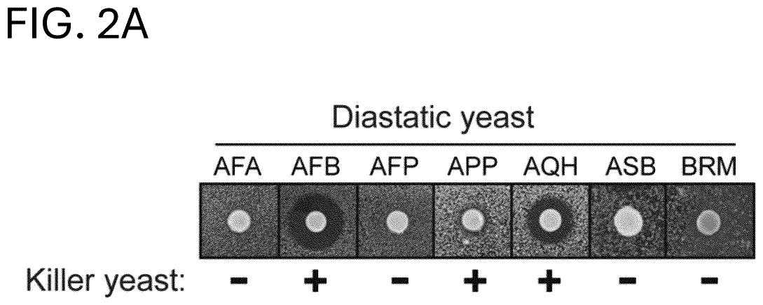

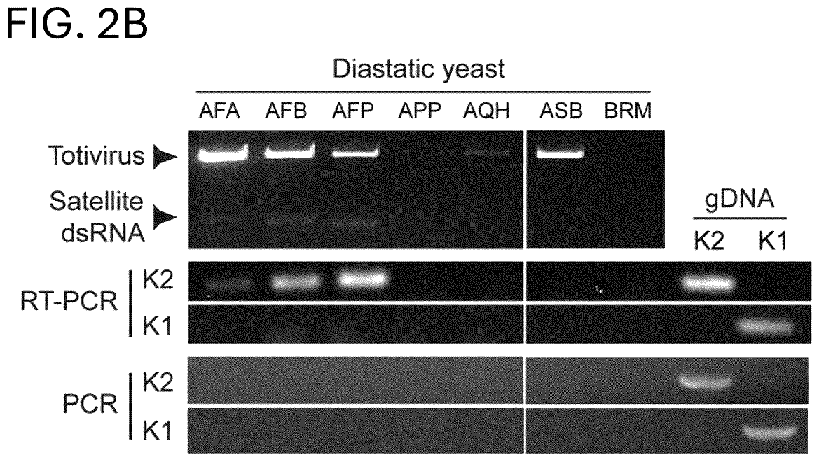

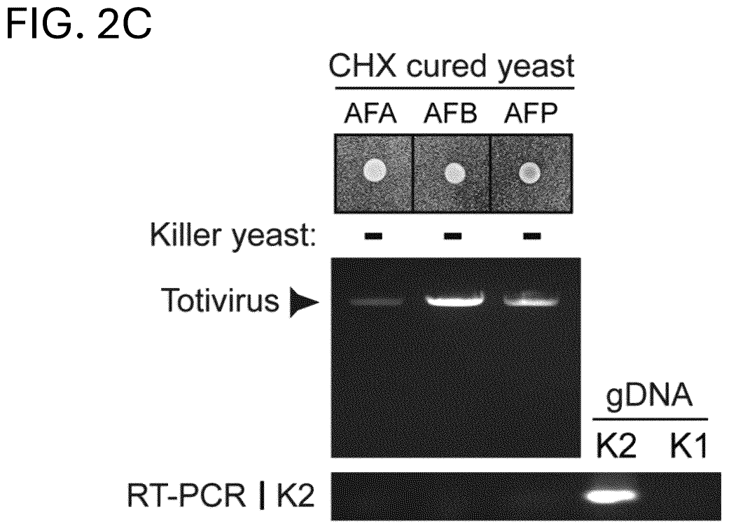

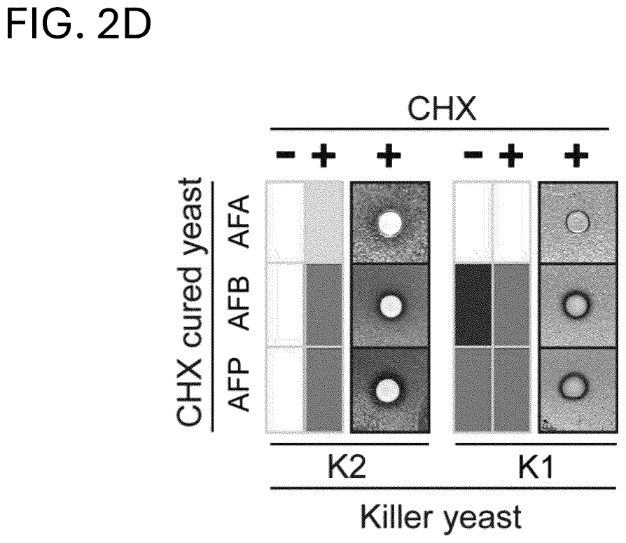

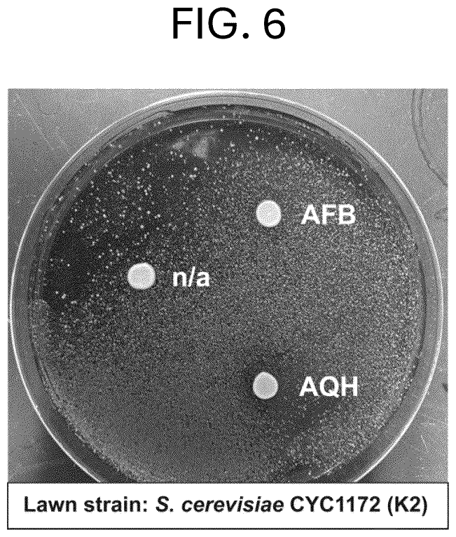

Killer toxin production by Saccharomyces yeasts is accompanied by immunity to the mature toxin. Thus, killer yeasts are immune to their own toxins. To determine whether the killer toxin-resistant diastatic yeasts had gained immunity due to killer toxin production, three K1-resistant diastatic yeasts and an additional ten strains resistant to K2 were used to challenge three lawns of S. cerevisiae known to be susceptible to K1 or K2. Only three diastatic strains were identified as killer yeasts (APP, AQH, and AFB) (FIGS. 2A & 5A-5B). None of these diastatic killer yeasts could inhibit the growth of the K2 killer yeast (FIG. 6), suggesting that these yeasts were likely K2 killer yeasts with immunity to K2. To determine whether killer toxin production was due to viruses and associated dsRNA satellites, each of the 14 killer toxin-resistant diastatic yeasts was subjected to analysis by cellulose chromatography to purify dsRNAs. This analysis revealed that five strains contained dsRNAs with sizes the same as totiviruses (~4.6 kb), and three strains harbored an additional satellite dsRNA (~1.5 kb) (FIGS. 2B & 5A-5B). Using total nucleic acid samples, reverse transcriptase PCR (RT-PCR) was used to detect the K2 killer toxin gene in the strains AFA, AFB, and AFP (FIG. 2B). K1 was not detected in any strains assayed by RT-PCR, and PCR alone could not amplify K1 or K2, indicating that the DNA genome does not encode these genes (FIG. 2B). Exposure to cycloheximide was used to cure the satellite dsRNAs from the killer yeasts AFA, AFB, and AFP, as determined by cellulose chromatography and RT-PCR (FIG. 2C). This curing treatment resulted in the loss of killer toxin production and increased susceptibility to K2, with K1 susceptibility remaining unchanged (FIG. 2D). These data show that while K1 resistance of diastatic yeasts was independent of dsRNAs, K2 resistance was due to the presence of M2 dsRNA satellites.

### Example 3

### A Variant K2 Killer Toxin Offers Broad-Spectrum Activity

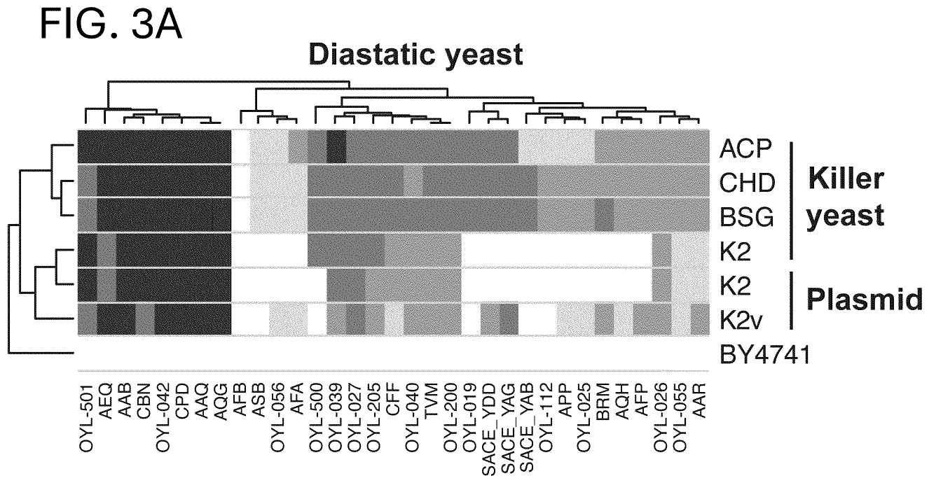

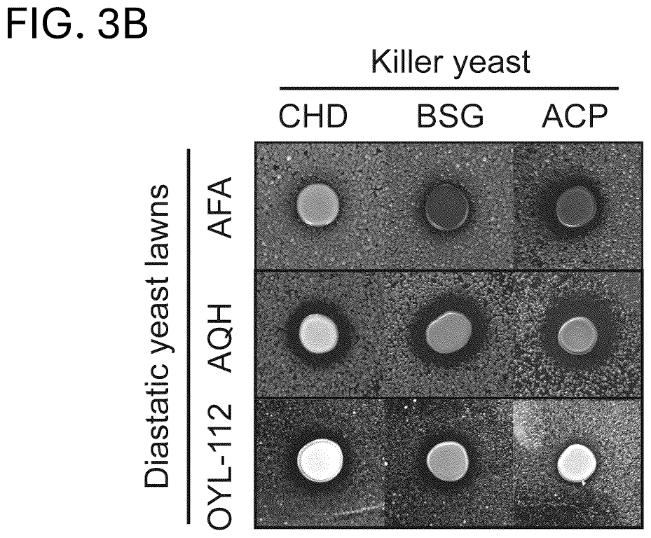

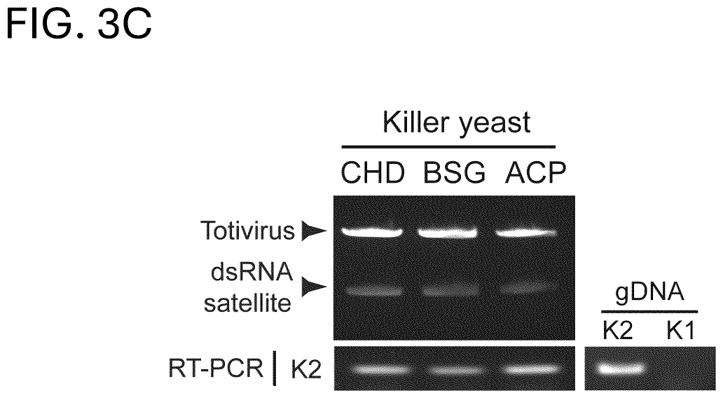

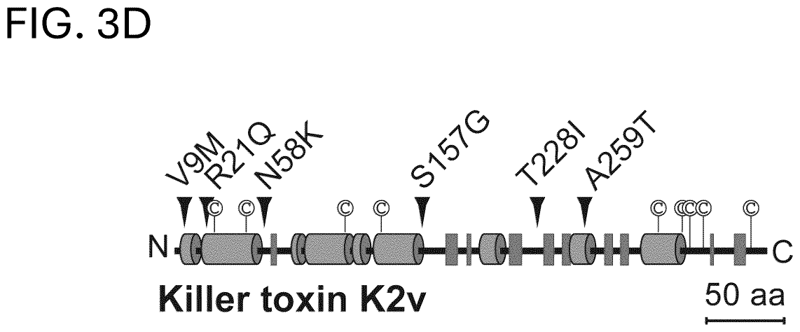

Several diastatic yeast strains were found to be resistant to K1 and K2 killer toxins (AFA, AQH, and OYL-112), and K1 and K74 (AFA and OYL-112). The diastatic strain OYL-112 was resistant to all canonical killer toxins. One hundred ninety-two previously identified and uncharacterized killer yeasts (see, Crabtree et al., “The prevalence of killer yeasts and double-stranded RNAs in the budding yeast Saccharomyces cerevisiae,” FEMS Yeast Res 23:foad046, 2023) were screened to determine whether there were Saccharomyces killer yeasts that could inhibit the growth of the killer toxin-resistant diastatic yeasts. In total, 32 killer yeasts were able to cause growth inhibition of K1 and K2-resistant diastatic yeast. Three strains of killer yeasts (CHD, BSG, and ACP) were judged the most effective at inhibiting the growth of resistant diastatic killer yeasts (FIG. 3A). These killer yeasts also inhibited the growth of all other diastatic yeast strains except AFB (FIG. 3B). These three novel killer yeasts were analyzed for dsRNAs using cellulose chromatography, which found that all three harbored totiviruses and satellite dsRNAs. RT-PCR was then used to confirm that these strains were K2 killer yeast (FIG. 3C). This result was surprising as this novel K2 variant could inhibit strains AFA, AQH, and OYL-112, which were all resistant to the canonical K2 toxin (FIGS. 1A-1C).

Purification and sequencing of the dsRNAs from CHD, BSG, and ACP confirmed that all three contained satellite dsRNAs with K2 killer toxin genes. These K2 genes had six non-synonymous mutations compared to canonical K2 (FIG. 3D). To distinguish this polymorphic toxin from canonical K2, it will be referred to as K2-variant (K2v) and the satellite dsRNA as M2v. K2v and K2 genes were introduced into a plasmid for expression in a non-killer laboratory strain of S. cerevisiae to directly compare the effect of the observed non-synonymous mutations on the spectrum of killer toxin activity. Comparing the galactose-induced expression of K2 and K2v from high copy plasmid expression, it was found that K2v had a broader spectrum of antifungal activity that could inhibit 78% of diastatic yeasts compared to K2, which inhibited only 50% (FIG. 3A). Galactose-induced expression of K2 was almost identical to the wild-type K2 killer yeast, but plasmid-expressed K2v inhibited less diastatic yeasts than the K2v killer yeasts CHD, BSG, and ACP (FIG. 3A). Plasmid-expressed K2v could not inhibit the diastatic K2 killer yeast AFB that harbored an M2 satellite dsRNA, suggesting that M2 immunity function could protect this strain from the K2v killer toxin. Surprisingly, K2v could inhibit the K2-resistant diastatic strains AFA and AFP that also harbored M2, indicating that K2 resistance is insufficient for K2v immunity. Overall, K2v is characterized as a variant K2 killer toxin with a broad-spectrum activity against diastatic yeast compared to canonical K2. Mutations in K2v likely caused changes in the killer toxin spectrum of activity that could inform the future development and application of K2 against diastatic yeasts.

### Example 4

### Prevention of Hyper-Attenuation by Diastatic Yeasts

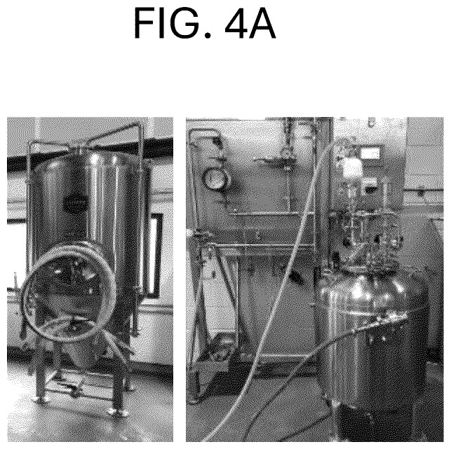

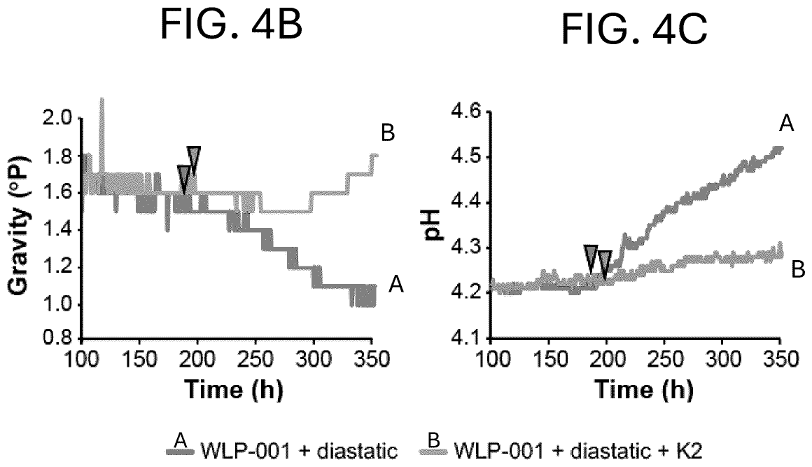

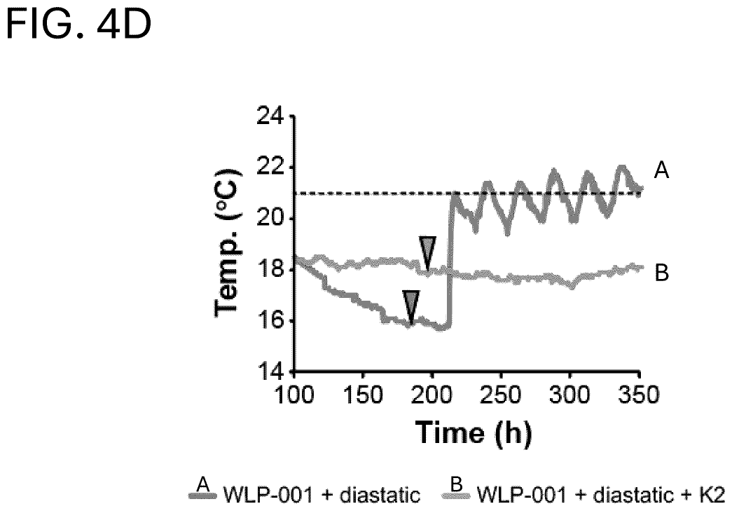

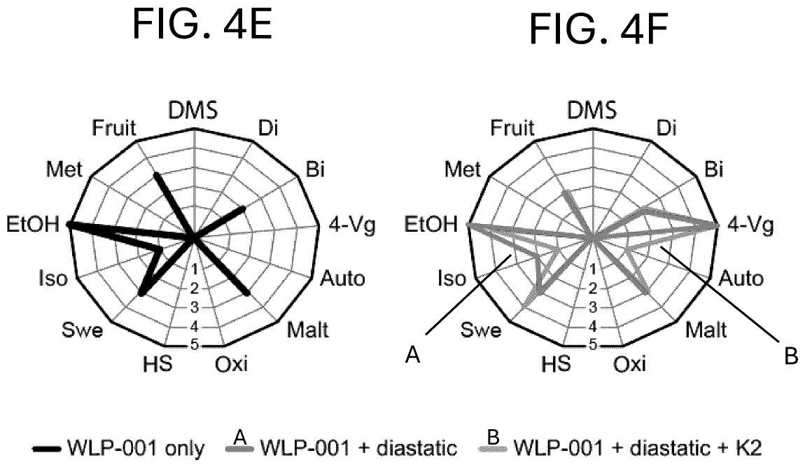

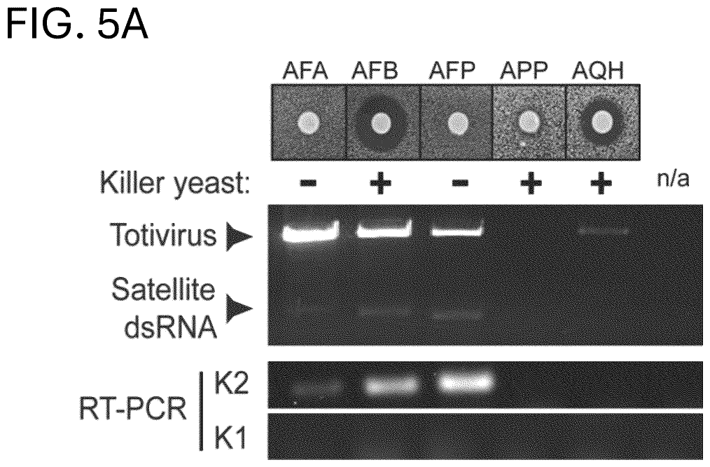

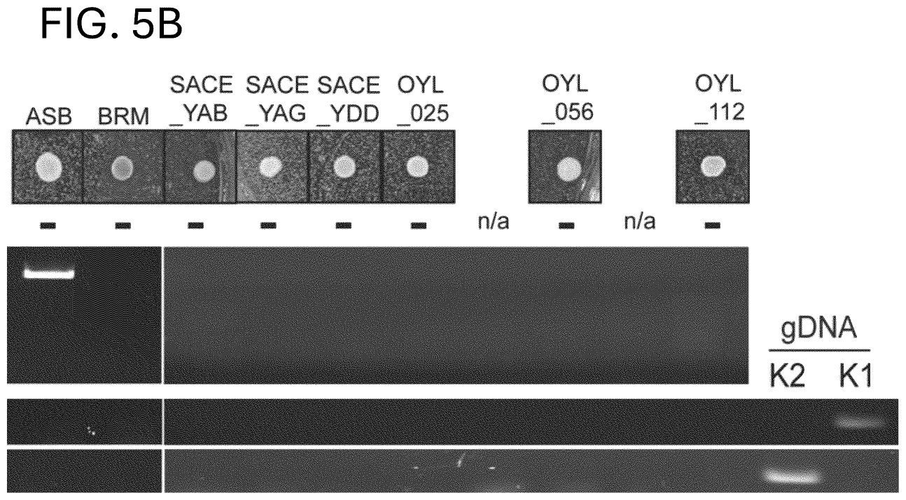

To determine whether it was possible to use killer yeasts to prevent hyperattenuation by diastatic yeasts, two 1,000-liter brewing trials were conducted using the brewing strain WLP-001 (FIG. 4A). Both fermentations proceeded normally in the first six days, with some variability in the gravity readings in the first 36-hour period due to the rapid evolution of CO₂. After approximately 100 hours of stable readings, fermentations were judged to have reached terminal gravity (~1.6° Plato (P)). In trial one, the diastatic POF+ yeast strain Belle Saison (Lallemand Inc.) was added to a final concentration of 5×10⁴ cells mL⁻¹. The addition of the diastatic yeast cells resulted in a rapid drop in gravity to 1.06° P (FIG. 4B) as well as an increase in pH (FIG. 4C) and temperature (FIG. 4D) before the trial was halted. This indicated that diastatic yeasts could ferment saccharides derived from the hydrolysis of residual starches and dextrins in the finished beer. For trial two, as the diastatic yeast Belle Saison was sensitive to the K2 killer toxin, remediation of a simulated contamination event was trialed by adding the K2 killer yeast strain Viva (Renaissance Yeast) that was chosen because of its routine use in the brewing industry (FIGS. 1A-1C). Moreover, Viva is a POF-strain with suitable alcohol tolerance, desirable ester profile, and reduced production of hydrogen sulfide and 4-vinyl guaiacol. Many of the characteristics of Viva are shared with the primary brewing strain WLP-001. The diastatic and killer yeast strains were added simultaneously to a final concentration of 5×10⁴ cells mL⁻¹. In contrast to trial one, the gravity in trial 2 dropped by only 0.08° P before recovering to 1.80° P at the end of the trial (FIG. 4B). The pH (FIG. 4C) and temperature (FIG. 4D) remained stable. The results suggest that it is possible to use killer yeasts to prevent hyperattenuation caused by diastatic yeasts.

To assess the effect of diastatic remediation on flavor profile, a sensory panel of trained cicerones performed a hedonic rating like/dislike and off-flavor evaluation on a 10-point scale. The yeast strain used in these trials (WLP-001) is commonly used in brewing and is characterized by a clean and fruity aroma (FIG. 4E). While it was evident that adding a K2 killer yeast prevented hyperattenuation, there was still a noticeable and undesirable flavor to the final brew. Specifically, while the beer produced from both diastatic trials maintained several desirable flavor characteristics (fruity/sweet/malty), they were very expressive of 4-vinyl guaiacol, which presented as clove or allspice, with a sensory score of 5 out of 10 in both fermentation trials (FIG. 4F). This off-flavor was present with or without adding the POF-K2 killer yeast strain (Viva) despite preventing hyperattenuation. Additionally, the trial with the K2 killer yeast had notes of an autolysis/meaty flavor that is attributed to the successful killing and lysis of the diastatic strain in this trial by the K2 killer toxin.

Proof-of-concept fermentation trials described here demonstrate that killer toxins effectively prevent diastatic hyperattenuation resulting from the growth of STA1+ Saccharomyces cerevisiae strains. However, remediation of diastatic contamination using K2 killer yeasts resulted in a noticeable and undesirable phenolic flavor, despite preventing hyperattenuation. The brewing strain WLP-001 was used in the primary fermentation for its clean aroma profile and POF-status; thus, the phenolic flavor after diastatic contamination was attributed to the addition of the diastatic POF+ Belle Saison yeast used in the trial (Table S4). In these trials, a high final concentration of diastatic yeast was added (5×10⁴ cells mL-1), considerably higher than the threshold for contamination in the brewing process (Burns et al., J Am Soc Brew Chem 79:167-180). Therefore, the large bolus of diastatic yeasts could be responsible for the undesirable flavor characteristics of the beer produced by these trials. Under more realistic scenarios with lower numbers of diastatic yeasts invading the brewing process, lower concentrations of the killer toxin in wort would likely be sufficient to prevent hyperattenuation and undesirable flavors. Indeed, killer toxins can trigger the apoptosis of susceptible yeasts at lower concentrations than those required for cell lysis (Sheppard et al., J Roy Soc Interface 16:20190064; Reiter et al., J Cell Biol 168:353-358).

### Example 5

### Exemplary Methods of Inhibiting Diastatic Contamination in Craft Breweries

Many craft breweries do not actively monitor for diastatic yeast contamination, therefore, engineering brewing strains that produce killer toxins during fermentation is one approach to safeguard against contamination. Killer toxin genes could be introduced into the yeast genome, for example, by selective breeding or direct genome editing. Alternatively, totiviruses and satellite dsRNAs encoding killer toxins could be introduced into existing brewing strains, for example by cytoduction (Young et al., J I Brewing 87:292-295). Yeasts are pitched into wort at high densities, thus killer toxins concentrations would likely increase rapidly during fermentation. Killer toxins present in the wort could prevent the invasion of diastatic yeasts at any downstream production stage.

S. cerevisiae acidifies wort during fermentation to a pH of ~4.2, which is optimal for killer toxin activity (Luksa et al., Mycoscience 57:51-57; Pfeiffer et al., Arch Microbiol 137:357-361). The stability of killer toxins in the finished beer remains to be investigated, but it is conceivable that at low pH and ambient temperatures, killer toxins would remain active during the packaging process and protect against diastatic contamination. Alternatively, killer yeast could also be used for “conditioning,” whereby yeast is added during packaging for natural carbonation. This would allow for killer toxin production in the packaged beer, protecting the finished product from diastatic yeast invasion. Regardless of the killer yeast application, important considerations include ensuring desirable fermentation profiles, flavor, and shelf life.

### Example 6

### Development of a New Yeast Donor Strain

Several strains of brewer's yeast capable of expressing killer toxins were developed through rare mating and cytoduction (Seki et al. “Construction of Killer Wine Yeast Strain” Applied and Environmental Microbiology 1985; 49: 1211-1215; Young “The genetic manipulation of killer character into brewing yeast.” J I Brewing. 1981; 87: 292-295; Hammond & Eckersley “Fermentation Properties of Brewing Yeast with Killer Character.” J Inst Brew. 1984; 90: 167-177). The expression of killer toxins by extrachromosomal double-stranded RNA (dsRNA) genetic elements (specifically dsRNA totiviruses and associated satellites) is useful for transferring the killer phenotype from laboratory strains to brewing strains without altering the DNA genome. This is beneficial because it does not alter the DNA genome of the brewing strain and, therefore, enables the maintenance of desirable qualities of fermentation.

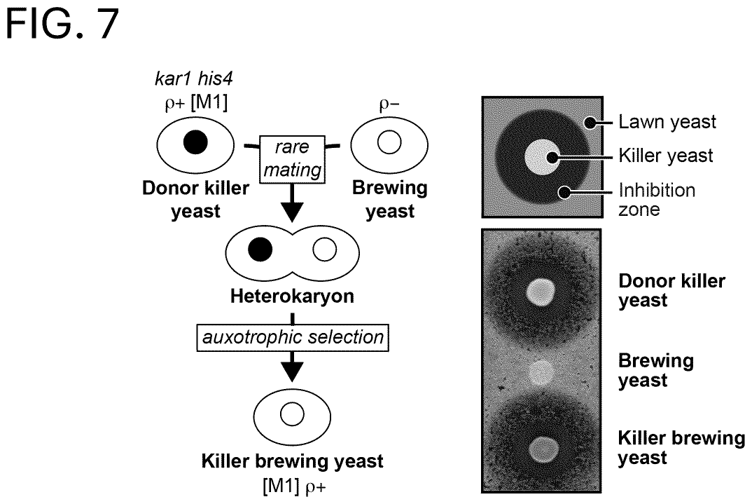

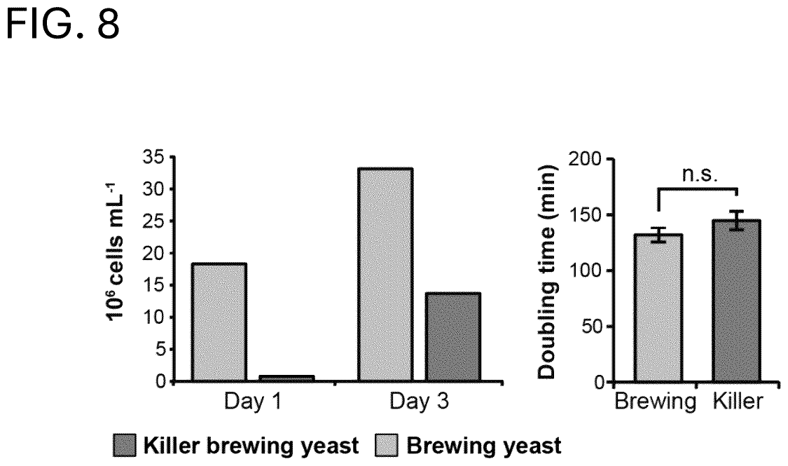

Success of this approach relies on strains of yeast that can act as donors of dsRNA satellites. Techniques of rare mating and cytoduction were piloted with a lab strain of S. cerevisiae 1368 (MATα his4 kar1 [L-A, M1]) (FIG. 7). This strain has a kar1 deficiency that prevents nuclear fusion after rare mating with a brewing strain, allowing the production of daughter cells with the DNA genome of the brewing strain but with cytoplasmically inherited dsRNA satellites. Donor strain 1368 harbors an M1 dsRNA satellite, MTA and the K1 killer phenotype was successfully transferred to four strains of well-known brewer's yeasts (WLP090 San Diego Super, WLP001 Cali Ale Yeast, CBC1 lalbrew barrel aging yeast, and OYL011 British Ale V). These killer brewing yeast can inhibit Saccharomyces cerevisiae var. diastaticus (diastaticus), a strain of yeast that possesses the STA1 gene, in co-culture on agar medium (FIG. 7). Importantly, killer brewing yeast can inhibit the growth of diastaticus in wort while retaining the growth characteristics of the parental brewing strain (FIG. 8).

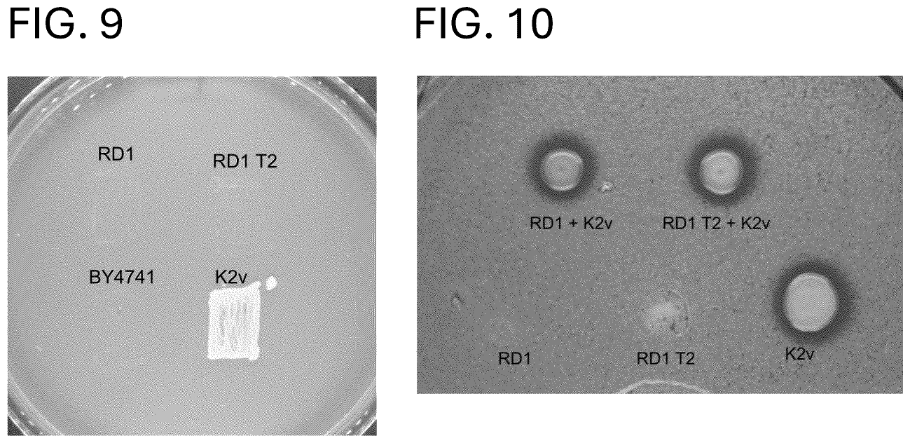

However, a major limitation of this approach is the lack of donor strains containing different dsRNA satellites that would allow the creation of brewing strains with different antifungal activities. To solve this problem, a novel donor strain of S. cerevisiae capable of receiving dsRNA satellites from different killer yeasts was created. Creating a library of different donors with various dsRNA satellites greatly expands the potential to enable brewer's yeasts to produce a wide array of antifungal killer toxins to fight contamination by undesirable species and strains of yeasts. The parental strain of the newly created strain is S. cerevisiae 2405 (genotype MATα his4 kar1), which lacks a dsRNA satellite and the L-A totivirus. Uracil auxotrophy was selected in this strain by growth on 5-fluoroorotic acid (5-FOA) as well as respiratory deficiency by growth on ethidium bromide. The resulting genotype of the strain was MATα his4 kar1 ura3 ρ− (rho negative) and was incapable of growing on complete media lacking uracil with glycerol as a sole carbon source (FIG. 9). This strain enables rare mating with non-laboratory strains of killer yeasts carrying distinct dsRNA satellites encoding different killer toxins. Such strains are then capable of transferring dsRNA satellites (and therefore the killer phenotype) to brewing strains. The new donor strain was designated “JM001.”

As a proof of principle, JM001 was rare-mated with a K2v expressing killer yeast (S. cerevisiae strain CHD), with selection for uracil auxotrophs (to select for the donor strain genotype and not that of CHD). Successful cytoduction was confirmed by the growth of auxotrophic yeasts on medium containing glycerol as the sole carbon source. As a final test, production of K2v was confirmed by observing a killer phenotype on YPD media (pH 4.6 with methylene blue) (FIG. 10). This new lab strain is now capable of donating M2v to brewers yeasts and has a genotype of MATα his4 kar1 ura3 [L-A, M2v].

As shown herein, the new strain of S. cerevisiae (JM001) can accept dsRNA satellites and be used to transfer the dsRNA satellites into brewing strains. This strain can be used to create a library of different donors with various dsRNA satellites to greatly expand the ability to move antifungal killer toxins into brewer yeast strains to fight contamination by undesirable species and strains of yeasts.

A biological deposit of S. cerevisiae strain JM001 has been made under the Budapest Treaty with the American Type Culture Collection (ATCC), 10801 University Boulevard, Manassas, VA 20110 USA, on ______, ATCC Accession No. ______. Access to the biological deposit will be available during the pendency of the application to the Commissioner of Patents and Trademarks and persons determined by the Commissioner to be entitled thereto upon request. All restrictions upon availability to the public will be irrevocably removed upon granting of the patent. The biological deposit will be maintained in the ATCC depository for a period of 30 years, or 5 years after the last request, or for the effective life of the patent, whichever is longer, and will be replaced if it becomes nonviable during that period.

It will be apparent that the precise details of the methods or compositions described herein may be varied or modified without departing from the spirit of the disclosure. We claim all such modifications and variations that fall within the scope and spirit of the claims below.

We claim:

1. A method of inhibiting diastatic yeast growth in a fermented beverage product, comprising:
inoculating the fermented beverage product or a pre-fermented form thereof with an engineered yeast comprising a heterologous nucleic acid molecule encoding a variant K2 (K2v) toxin, wherein the K2v toxin comprises an amino acid sequence having at least 90% sequence identity to SEQ ID NO: 1, and comprises a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259,
wherein the amino acid positions are according to a reference K2v sequence set forth as SEQ ID NO: 1.

2. The method of claim 1, wherein the fermented beverage product is beer.

3. The method of claim 1, wherein the pre-fermented form of the fermented beverage product is inoculated with the engineered yeast.

4. The method of claim 1, wherein the K2v toxin comprises:
(i) at least 95% sequence identity to SEQ ID NO: 1;
(ii) at least 98% sequence identity to SEQ ID NO: 1; and/or
(iii) at least 99% sequence identity to SEQ ID NO: 1.

5. The method of claim 1, wherein the K2v toxin comprises or consists of SEQ ID NO: 1.

6. The method of claim 1, wherein the heterologous nucleic acid molecule comprises:
(i) at least 90% sequence identity to SEQ ID NO: 2;
(ii) at least 95% sequence identity to SEQ ID NO: 2; and/or
(iii) at least 98% sequence identity to SEQ ID NO: 2.

7. The method of claim 1, wherein the heterologous nucleic acid molecule comprises SEQ ID NO: 2 or a degenerate variant of SEQ ID NO: 2.

8. The method of claim 1, wherein the heterologous nucleic acid molecule is comprised on a vector.

9. The method of claim 8, wherein the vector is a viral vector.

10. The method of claim 9, wherein the viral vector is a M2 satellite virus vector.

11. The method of claim 1, wherein the heterologous nucleic acid molecule is cytoplasmic double-stranded RNA (dsRNA).

12. The method of claim 8, wherein the vector is a plasmid.

13. The method of claim 12, wherein the heterologous nucleic acid molecule comprises:
(i) at least 90% sequence identity to SEQ ID NO: 3 or SEQ ID NO: 18;
(ii) at least 95% sequence identity to SEQ ID NO: 3 or SEQ ID NO: 18; and/or
(iii) at least 98% sequence identity to SEQ ID NO: 3 or SEQ ID NO: 18.

14. The method of claim 13, wherein the heterologous nucleic acid molecule comprises or consists of SEQ ID NO: 3 or SEQ ID NO: 18, or a degenerate variant of SEQ ID NO: 3 or SEQ ID NO: 18.

15. The method of claim 1, wherein the engineered yeast is S. cerevisiae modified to comprise the heterologous nucleic acid molecule encoding the variant K2 (K2v) toxin.

16. The method of claim 15, wherein the S. cerevisiae is strain OYL090 (Omega Yeast San Diego Super), WLP001 (White Laps California Ale Yeast), CBC1 (Lallemand conditioning yeast), WLP028 (White Labs Edinburgh Scottish Ale), WLP940 (White Labs Mexican Lager Yeast), L28 (Imperial Yeast Urkel Czech lager yeast), OYL061 (Omega Yeast Voss Kveik), or OYL011.

17. The method of claim 1, wherein the diastatic yeast is resistant to canonical K2 toxin set forth as SEQ ID NO: 4.

18. The method of claim 1, further comprising selecting the fermented beverage product as having or suspected of having diastatic yeast contamination before inoculating the fermented beverage product or the pre-fermented form thereof with the engineered yeast.

19. The method of claim 1, wherein the fermented beverage product is beer and the method comprises the steps of:
(i) selecting a wort that has or is suspected of having contamination with a diastatic yeast that is resistant to canonical K2 toxin set forth as SEQ ID NO: 4;
(ii) inoculating the wort with S. cerevisiae expressing a variant K2 (K2v) toxin set forth as SEQ ID NO: 1; and
(iii) fermenting the wort to produce beer;
thereby inhibiting diastatic yeast growth in the beer.

20. An engineered yeast comprising a recombinant vector encoding a variant K2 (K2v) toxin, wherein:
the K2v toxin comprises an amino acid sequence having at least 90% sequence identity to SEQ ID NO: 1, and comprises a methionine (M) at amino acid position 9, a glutamine (Q) at amino acid position 21, a lysine (K) at amino acid position 58, a glycine (G) at amino acid position 157, an isoleucine (I) at amino acid position 228, and a threonine (T) at amino acid position 259, wherein the amino acid positions are according to a reference K2v sequence set forth as SEQ ID NO: 1, and
the recombinant vector comprises a nucleotide sequence at least 90% identical to SEQ ID NO: 3.

21. A method of inhibiting diastatic yeast growth in a fermented beverage product, comprising inoculating the fermented beverage product, or a pre-fermented form thereof, with the engineered yeast of claim 20.

22. A Saccharomyces cerevisiae strain designated JM001, wherein a representative sample of cells of Saccharomyces cerevisiae strain JM001 has been deposited under ATCC Accession No. PTA-______.


SEQUENCE LISTING (18 sequences)

SEQ ID NO: 1 - protein, 362 aa, Saccharomyces cerevisiae
MKETTTSLMQDELTLGEPATQARMCVRLLRFFIGLTITAFIIAACIIKSATGGSGYSKAVAVRGEADTPSTIVGQLVERGGFQAWAVGAGIYLFAKIAYDTSKVTAAVCNPEALIAITSYVAYAPTLCAGAYVIGAMSGAMSAGLALYAGYKGWQWGGPGGMAEREDVASFYSPLLNNTLYVGGDHTADYDSELATILGSVYNDVVHLGVYYDNSTGIVKRDSRPSMISWTVLHDNMMITSYHRPDQLGAAATAYKAYTTNTTRVGKRQDGEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 2 - DNA, 1029 bp, Saccharomyces cerevisiae
  source 1..1029: mol_type = other DNA
atgaaagagactaccaccagcctgatgcaagacgagctgacactaggtgagccggccacccaagcgaggatgtgcgtacgtctattacgttttttcataggtctgactataaccgcatttattatagcagcctgtattattaaaagtgcgacaggcggttcgggatattctaaagcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcgggccttgctctgtatgccggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgggcgcagccgcgacagcctacaaagcttataccacaaacacaacacgggtcggtaagaggcaggacggtgagtgggtgtcatactcggtctacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatg

SEQ ID NO: 3 - DNA, 7770 bp, synthetic construct
  source 1..7770: mol_type = other DNA
gacgaaagggcctcgtgatacgcctatttttataggttaatgtcatgataataatggtttcttagtatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggattctagaactagtggatcccccatcacaagtttgtacaaaaaagcaggctccgaattcgcccttatgaaagagactaccaccagcctgatgcaagacgagctgacactaggtgagccggccacccaagcgaggatgtgcgtacgtctattacgttttttcataggtctgactataaccgcatttattatagcagcctgtattattaaaagtgcgacaggcggttcgggatattctaaagcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcgggccttgctctgtatgccggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgggcgcagccgcgacagcctacaaagcttataccacaaacacaacacgggtcggtaagaggcaggacggtgagtgggtgtcatactcggtctacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatggtcggtgaggcctacttctatgcctatggtggggttgatggtgaatgtgacagcggctagaagggcgaattcgacccagcttttttcttgtacaaagtggtgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaaaggtggtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 4 - protein, 362 aa, Saccharomyces cerevisiae
MKETTTSLVQDELTLGEPATRARMCVRLLRFFIGLTITAFIIAACIIKSATGGSGYSNAVAVRGEADTPSTIVGQLVERGGFQAWAVGAGIYLFAKIAYDTSKVTAAVCNPEALIAITSYVAYAPTLCAGAYVIGAMSGAMSAGLALYAGYKGWQWSGPGGMAEREDVASFYSPLLNNTLYVGGDHTADYDSELATILGSVYNDVVHLGVYYDNSTGIVKRDSRPSMTSWTVLHDNMMITSYHRPDQLGAAATAYKAYATNTTRVGKRQDGEWVSYSVYGENVDYERYPVAHLQEEADACYESLGNMITSQVQPCTQRECYAMDQKVCAAVGFSSDAGVNSAMVGEAYFYAYGGVDGECDSG

SEQ ID NO: 5 - DNA, 1089 bp, Saccharomyces cerevisiae
  source 1..1089: mol_type = other DNA
atgaaagagactaccaccagcctggtgcaagacgagctgacactaggtgagccggccacccgagcaaggatgtgcgtacgtctattacgttttttcataggtctgactataaccgcatttattatagcagcctgtattattaaaagtgcgacaggcggttcgggatattctaatgcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcgggccttgctctgtatgccggttacaaaggatggcagtggagcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgacctcatggacggtgttgcatgacaacatgatgataacatcataccataggccagaccagctgggcgcagccgcgacagcctacaaagcttatgccacaaacacaacacgggtcggtaagaggcaggacggtgagtgggtgtcatactcggtctacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatggtcggtgaggcctacttctatgcctatggtggggttgatggtgaatgtgacagcggctag

SEQ ID NO: 6 - DNA, 25 bp, synthetic construct
  source 1..25: mol_type = other DNA
tgtcggctaatggtaacctgtatgg

SEQ ID NO: 7 - DNA, 26 bp, synthetic construct
  source 1..26: mol_type = other DNA
gtcacagccttcaaagtcattattgg

SEQ ID NO: 8 - DNA, 25 bp, synthetic construct
  source 1..25: mol_type = other DNA
gtggcctctttttattcaccactcc

SEQ ID NO: 9 - DNA, 25 bp, synthetic construct
  source 1..25: mol_type = other DNA
gtctcgaatccctcttgacaattcc

SEQ ID NO: 10 - DNA, 21 bp, synthetic construct
  source 1..21: mol_type = other DNA
atgaaagagactaccaccagc

SEQ ID NO: 11 - DNA, 22 bp, synthetic construct
  source 1..22: mol_type = other DNA
gatcggcgacagtgtaagtggt

SEQ ID NO: 12 - DNA, 28 bp, synthetic construct
  source 1..28: mol_type = other DNA
atgaaagagactaccaccagcctgatgc

SEQ ID NO: 13 - DNA, 28 bp, synthetic construct
  source 1..28: mol_type = other DNA
ctagccgctgtcacattcaccatcaacc

SEQ ID NO: 14 - DNA, 32 bp, synthetic construct
  source 1..32: mol_type = other DNA
gaaaaataaagaaatgacgaagccaacccaag

SEQ ID NO: 15 - DNA, 26 bp, synthetic construct
  source 1..26: mol_type = other DNA
gagttatcgcatcagaggtcagacac

SEQ ID NO: 16 - DNA, 17 bp, synthetic construct
  source 1..17: mol_type = other DNA
actggccgtcgttttac

SEQ ID NO: 17 - DNA, 17 bp, synthetic construct
  source 1..17: mol_type = other DNA
gtcatagctgtttcctg

SEQ ID NO: 18 - DNA, 6142 bp, synthetic construct
  source 1..6142: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactatagggcgaattggagctctagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaaccccggattctagaactagtggatcccccgtacaaaaaagcaggctccgaattcgcccttatgaaagagactaccaccagcctgatgcaagacgagctgacactaggtgagccggccacccaagcgaggatgtgcgtacgtctattacgttttttcataggtctgactataaccgcatttattatagcagcctgtattattaaaagtgcgacaggcggttcgggatattctaaagcagttgctgttcggggagaagcggacaccccttccacaattgtgggccagctcgtcgagcgtggcggcttccaagcttgggcagtgggggctggtatctatttgtttgccaagatagcatatgatacatctaaggttaccgcagctgtatgtaatccggaggcgctcattgctatcacatcgtatgtggcatatgcccctacactgtgtgctggtgcatacgttattggtgccatgagtggggcaatgtcggcgggccttgctctgtatgccggttacaaaggatggcagtggggcggccccgggggcatggcagagagagaggacgtggcctctttttattcaccactcctgaacaacactctgtacgtgggtggggaccacactgcagactacgacagtgaattggctactatattaggtagcgtatataatgatgtggtccacctgggggtgtattacgataacagcactggaattgtcaagagggattcgagacctagcatgatctcatggacggtgttgcatgacaacatgatgataacatcataccataggccagaccagctgggcgcagccgcgacagcctacaaagcttataccacaaacacaacacgggtcggtaagaggcaggacggtgagtgggtgtcatactcggtctacggtgagaatgttgactatgaaagataccctgtagcacatctgcaagaggaggccgacgcgtgttacgagagtttaggtaatatgattacgagccaggtacagccctgtactcagagagaatgttatgctatggatcagaaagtatgcgcagctgtcggcttctcatcagatgcgggtgttaactccgcaatgaagggcgaattcgacccagcgatgggctgcaggaattcgatatcaagcttatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc